(19) United States
(12) Patent Application Publication
(10) Pub. No.: US 2019/0216829 A1
(43) Pub. Date: Jul. 18, 2019

(54) Anticancer Compositions

(71) Applicant: ARAGON PHARMACEUTICALS, INC., San Diego, CA (US)

(72) Inventors: Johny BERTELS, Beerse (BE); Jurgen MENSCH, Beerse (BE)

(21) Appl. No.: 16/306,802

(22) PCT Filed: May 16, 2017
(86) PCT No.: PCT/US2017/032815

§ 371 Date: Dec. 3, 2018

(30) Foreign Application Priority Data
Jun. 3, 2016 (EP) 16172968.6

(51) Int. Cl. A61K 31/58; A61K 31/4439; A61K 31/573; A61K 31/57; A61K 9/20; A61K 9/00

(52) U.S. Cl. CPC A61K 31/58; A61K 31/4439; A61K 31/573; A61K 9/2095; A61K 9/2086; A61K 9/2054; A61K 9/0053; A61K 31/57

(57) ABSTRACT

The present invention concerns pharmaceutical formulations of abiraterone acetate and ARN-509, which can be administered to a mammal, in particular a human, suffering from an androgen receptor (AR)-related disease or condition, in particular cancer, more in particular prostate cancer, including but not limited to castration-resistant prostate cancer, metastatic castration resistant prostate cancer, chemotherapy-naive metastatic castration resistant prostate cancer, biochemically relapsed hormone sensitive prostate cancer, or high-risk, non-metastatic castration-resistant prostate cancer. In one aspect, these formulations comprise abiraterone acetate and a solid dispersion of ARN-509 and a polymer selected from HPMCAS, a poly(meth)acrylate copolymer, and mixtures thereof. In one aspect, these formulations comprise a granulate of abiraterone acetate and a solid dispersion of ARN-509 and a polymer selected from HPMC AS, a poly(meth)acrylate copolymer, and mixtures thereof.

The present invention concerns pharmaceutical formulations of abiraterone acetate and ARN-509, which can be administered to a mammal, in particular a human, suffering from an androgen receptor (AR)-related disease or condition, in particular cancer, more in particular prostate cancer, including but not limited to castration-resistant prostate cancer, metastatic castration resistant prostate cancer, chemotherapy-naive metastatic castration resistant prostate cancer, biochemically relapsed hormone sensitive prostate cancer, or high-risk, non-metastatic castration-resistant prostate cancer. In one aspect, these formulations comprise abiraterone acetate and a solid dispersion of ARN-509 and a polymer selected from HPMCAS, a poly(meth)acrylate copolymer, and mixtures thereof. In one aspect, these formulations comprise a granulate of abiraterone acetate and a solid dispersion of ARN-509 and a polymer selected from HPMCAS, a poly(meth)acrylate copolymer, and mixtures thereof.

The present invention concerns pharmaceutical formulations of abiraterone acetate and ARN-509, which can be administered to a mammal, in particular a human, suffering from an androgen receptor (AR)-related disease or condition, in particular cancer, more in particular prostate cancer, including but not limited to castration-resistant prostate cancer, metastatic castration resistant prostate cancer, chemotherapy-naive metastatic castration resistant prostate cancer, biochemically relapsed hormone sensitive prostate cancer, or high-risk, non-metastatic castration-resistant prostate cancer. In one aspect, these formulations comprise abiraterone acetate and a solid dispersion of ARN-509 and a polymer selected from HPMCAS, a poly(meth)acrylate copolymer, and mixtures thereof. In one aspect, these formulations comprise a granulate of abiraterone acetate and a solid dispersion of ARN-509 and a polymer selected from HPMCAS, a poly(meth)acrylate copolymer, and mixtures thereof. In one aspect, the solid dispersion of ARN-509 and a polymer selected from HPMCAS, a poly(meth)acrylate copolymer, and mixtures thereof is obtainable, in particular is obtained, by melt-extruding a mixture comprising ARN-509 and a polymer selected from HPMCAS, a poly(meth)acrylate copolymer, and mixtures thereof and optionally subsequently milling said melt-extruded mixture. In one aspect, the solid dispersion of ARN-509 and a polymer selected from HPMCAS, a poly(meth)acrylate copolymer, and mixtures thereof is obtainable, in particular is obtained, by spray drying a mixture comprising ARN-509 and a polymer selected from HPMCAS, a poly(meth)acrylate copolymer, and mixtures thereof in a suitable solvent.

The pharmaceutical formulations of the present invention provide improved stability or improved shelf life. With the formulations of the present invention the pill burden for the patient, in particular the cancer patient, can be reduced, and hence therapy adherence and therapy efficiency can be improved. The manufacture of the pharmaceutical formulations, in particular in the form of a tablet or a capsule, does not require a flow or density property improving step such as a roller compaction step notwithstanding the presence of a solid dispersion in the formulations of the present invention.

### FIGURES

FIG. 1: XRD pattern of ARN-509 Form B.

FIG. 2: IR spectrum of ARN-509 Form B.

FIG. 3: DSC curve of ARN-509 Form B.

### DETAILED DESCRIPTION

ARN-509 (apalutamide) is a potent and specific antagonist of the androgen receptor (AR). ARN-509's mechanism of action is antagonism of androgen receptor signaling through inhibition of AR nuclear translocation and DNA binding to androgen response elements.

The actions of androgens with androgen receptors have been implicated in a number of diseases or conditions, such as androgen dependent cancers, virilization in women, and acne, among others. Compounds that diminish the effects of androgens with androgen receptors and/or lower the concentrations of androgen receptors find use in the treatment of diseases or conditions in which androgen receptors play a role.

AR-related diseases or conditions include, but are not limited to, benign prostate hyperplasia, hirsutism, acne, adenomas and neoplasias of the prostate, benign or malignant tumor cells containing the androgen receptor, hyperpilosity, seborrhea, endometriosis, polycystic ovary syndrome, androgenic alopecia, hypogonadism, osteoporosis, suppression of spermatogenesis, libido, cachexia, anorexia, androgen supplementation for age related decreased testosterone levels, prostate cancer, breast cancer, endometrial cancer, uterine cancer, hot flashes, Kennedy's disease muscle atrophy and weakness, skin atrophy, bone loss, anemia, arteriosclerosis, cardiovascular disease, loss of energy, loss of well-being, type 2 diabetes, and abdominal fat accumulation. Given the central role of AR in prostate cancer development and progression, ARN-509 is useful for the treatment of cancer, in particular prostate cancer, including but not limited to castration-resistant prostate cancer, metastatic castration resistant prostate cancer, chemotherapy-naive metastatic castration resistant prostate cancer, biochemically relapsed hormone sensitive prostate cancer, or high-risk, non-metastatic castration-resistant prostate cancer.

The chemical structure of ARN-509 is:

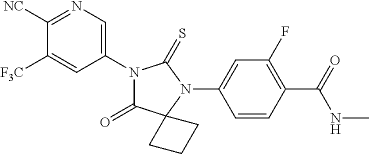 [SMILES: CNC(=O)c1ccc(N2C(=S)N(c3cnc(C#N)c(C(F)(F)F)c3)C(=O)C23CCC3)cc1F]

ARN-509 or 4-[7-(6-cyano-5-trifluoromethylpyridin-3-yl)-8-oxo-6-thioxo-5,7-diazaspiro[3.4]oct-5-yl]-2-fluoro-N-methylbenzamide is currently in clinical development as a nonaqueous, lipid-based solution that is filled into softgel capsules, each containing 30 mg ARN-509. The daily dose being studied is 240 mg/day by oral administration (or 8 softgel capsules). It has been found that in use, the softgel capsules containing ARN-509 have a shelf life of only 6 months and need cold chain storage.

Abiraterone acetate of formula:

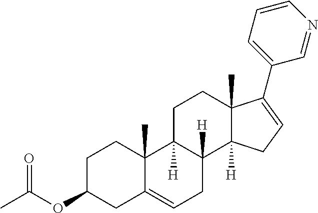

is a potent selective, orally active inhibitor of the key enzyme in testosterone synthesis, 17α-hydroxylase-C17,20-lyase, also known as steroid 17α-monooxygenase inhibitor or Human Cytochrome P45017α. Suppression of testosterone synthesis has been demonstrated with abiraterone acetate in patients with prostate cancer. The compound was first disclosed in WO-A-93/20097.

Abiraterone acetate is approved in post-chemotherapy and chemotherapy-naïve metastatic castration-resistant prostate cancer (mCRPC) and is currently in clinical development for high-risk metastatic hormone-sensitive prostate cancer (mHSPC). Abiraterone acetate is currently on the market as a 250 mg oral tablet for administration of four tablets once daily or as a 500 mg oral tablet for administration of two tablets once daily.

An aspect of the invention relates to pharmaceutical formulations, in particular solid pharmaceutical formulations, more in particular solid pharmaceutical formulations for oral adminstration of abiraterone acetate and ARN-509, such as for example tablets or capsules, where such formulations have an improved stability, a longer shelf life, or provide for a reduced pill burden for the patient, in particular the cancer patient. The pharmaceutical formulations of the present invention provide a means to increase therapy adherence and therapy efficiency. The pharmaceutical formulations of the invention provide for the avoidance of a flow property improving step, such as a roller compaction step, in their manufacturing process.

An aspect of the invention is a pharmaceutical formulation comprising a pharmaceutically acceptable carrier, abiraterone acetate and a solid dispersion, said solid dispersion comprising ARN-509 and a polymer selected from HPMCAS, a poly(meth)acrylate copolymer, and mixtures thereof.

HPMCAS or hydroxypropyl methylcellulose acetate succinate or hypromellose acetate succinate (CAS number 71138-97-1) is a mixture of acetic acid and monosuccinic acid esters of hydroxypropylmethyl cellulose (IUPAC name: cellulose, 2-hydroxypropyl methyl ether, acetate, hydrogen butanedioate). Different grades are available differentiated based on degree/ratio of substitution (acetyl content, succinoyl content) and particle size (micronized and granular). In an aspect of the invention, the HPMCAS in the dispersions with ARN-509 is HPMCAS LG (granular grade) or HPMCAS LF (micronized grade) (Shin-Etsu Chemical Co., Ltd), in particular HPMCAS LG. A preferred grade of HPMCAS in the solid dispersions of the pharmaceutical formulations of the invention is HPMCAS LG, because of its better and safer handling properties.

Copolymers derived from esters of acrylic and methacrylic acid (poly(meth)acrylates) are known in the industry as Eudragit®. Eudragit® is the brand name for a diverse range of poly(meth)acrylate-based copolymers. Different grades are available. In an aspect of the invention, the Eudragit® in the dispersions with ARN-509 is Eudragit® L 100-55 which contains an anionic copolymer based on methacrylic acid and ethyl acrylate (CAS number 25212-88-8; Chemical/IUPAC name: Poly(methacrylic acid-co-ethyl acrylate) 1:1) (Evonik Industries). In an aspect of the invention, the Eudragit® in the dispersions with ARN-509 is Eudragit® E 100 which is a cationic copolymer based on dimethylaminoethyl methacrylate, butyl methacrylate, and methyl methacrylate (CAS number 24938-16-7; Chemical/IUPAC name: Poly(butyl methacrylate-co-(2-dimethylaminoethyl) methacrylate-co-methyl methacrylate) 1:2:1 (Evonik Industries).

In an aspect of the invention, the weight-by-weight ratio of ARN-509:polymer in the solid dispersion of the pharmaceutical formulations as described herein is in the range from 1:1 to 1:10, preferably from 1:1 to 1:5, more preferably from 1:1 to 1:3 or from 1:2 to 1:3. In an aspect of the invention, the weight-by-weight ratio of ARN-509:polymer is 1:1. In an aspect of the invention, the weight-by-weight ratio of ARN-509:polymer is 1:2. In an aspect of the invention, the weight-by-weight ratio of ARN-509:polymer is 1:3.

In an aspect of the invention, the polymer in the solid dispersion is HPMCAS and the weight-by-weight ratio of ARN-509:HPMCAS is 1:1, 1:2 or 1:3.

In an aspect of the invention, the polymer in the solid dispersion is HPMCAS LG and the weight-by-weight ratio of ARN-509:HPMCAS LG is 1:1, 1:2 or 1:3.

In an aspect of the invention, the polymer in the solid dispersion is HPMCAS LF and the weight-by-weight ratio of ARN-509:HPMCAS LF is 1:1, 1:2 or 1:3.

In an aspect of the invention, the polymer in the solid dispersion is a poly(meth)acrylate copolymer and the weight-by-weight ratio of ARN-509:poly(meth)acrylate copolymer is 1:1, 1:2 or 1:3.

In an aspect of the invention, the polymer in the solid dispersion is Eudragit® L 100-55 and the weight-by-weight ratio of ARN-509:Eudragit® L 100-55 is 1:1, 1:2 or 1:3.

In an aspect of the invention, the polymer in the solid dispersion is Eudragit® E 100 and the weight-by-weight ratio of ARN-509:Eudragit® E 100 is 1:1, 1:2 or 1:3.

In an aspect of the invention, the polymer in the solid dispersion is a mixture of a poly(meth)acrylate copolymer and HPMCAS and the weight-by-weight ratio of ARN-509:(poly(meth)acrylate copolymer and HPMCAS) is 1:1, 1:2 or 1:3.

In an aspect of the invention, the polymer in the solid dispersion is a mixture of Eudragit® L 100-55 and HPMCAS LG and the weight-by-weight ratio of ARN-509:(Eudragit® L 100-55 and HPMCAS LG) is 1:1, 1:2 or 1:3.

In an aspect of the invention, the polymer in the solid dispersion is a mixture of Eudragit® E 100 and HPMCAS LG and the weight-by-weight ratio of ARN-509:(Eudragit® E 100 and HPMCAS LG) is 1:1, 1:2 or 1:3.

In an aspect of the invention, the polymer in the solid dispersion is a mixture of Eudragit® L 100-55 and HPMCAS LF and the weight-by-weight ratio of ARN-509:(Eudragit® L 100-55 and HPMCAS LF) is 1:1, 1:2 or 1:3.

In an aspect of the invention, the polymer in the solid dispersion is a mixture of Eudragit® E 100 and HPMCAS LF and the weight-by-weight ratio of ARN-509:(Eudragit® E 100 and HPMCAS LF) is 1:1, 1:2 or 1:3.

In an aspect of the invention, the polymer in the solid dispersion is a mixture of a poly(meth)acrylate copolymer and HPMCAS and the weight-by-weight ratio of poly(meth)acrylate copolymer to HPMCAS ranges from 5:95 to 95:5, in particular from 10:90 to 90:10, more in particular from 25:75 to 75:25. Preferably, the weight-by-weight ratio of poly(meth)acrylate copolymer to HPMCAS in the solid dispersion of the pharmaceutical formulations as described herein is 50:50.

An aspect of the invention is a pharmaceutical formulation comprising a pharmaceutically acceptable carrier, abiraterone acetate and a solid dispersion, said solid dispersion comprising ARN-509 and HPMCAS. An aspect of the invention is a pharmaceutical formulation comprising a pharmaceutically acceptable carrier, abiraterone acetate and a solid dispersion, said solid dispersion consisting of ARN-509 and HPMCAS. In an aspect, the weight-by-weight ratio of ARN-509:HPMCAS is 1:1, 1:2 or 1:3.

An aspect of the invention is a pharmaceutical formulation comprising a pharmaceutically acceptable carrier, abiraterone acetate and a solid dispersion, said solid dispersion comprising ARN-509 and HPMCAS LG. An aspect of the invention is a pharmaceutical formulation comprising a pharmaceutically acceptable carrier, abiraterone acetate and a solid dispersion, said solid dispersion consisting of ARN-509 and HPMCAS LG. In an aspect, the weight-by-weight ratio of ARN-509:HPMCAS LG is 1:1, 1:2 or 1:3.

An aspect of the invention is a pharmaceutical formulation comprising a pharmaceutically acceptable carrier, abiraterone acetate and a solid dispersion, said solid dispersion comprising ARN-509 and HPMCAS LF. An aspect of the invention is a pharmaceutical formulation comprising a pharmaceutically acceptable carrier, abiraterone acetate and a solid dispersion, said solid dispersion consisting of ARN-509 and HPMCAS LF. In an aspect, the weight-by-weight ratio of ARN-509:HPMCAS LF is 1:1, 1:2 or 1:3.

An aspect of the invention is a pharmaceutical formulation comprising a pharmaceutically acceptable carrier, abiraterone acetate and particles comprising a solid dispersion, said solid dispersion comprising ARN-509 and HPMCAS. An aspect of the invention is a pharmaceutical formulation comprising a pharmaceutically acceptable carrier, abiraterone acetate and particles comprising a solid dispersion, said solid dispersion consisting of ARN-509 and HPMCAS. In an aspect, the weight-by-weight ratio of ARN-509:HPMCAS is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles consisting of a solid dispersion, said solid dispersion comprising ARN-509 and HPMCAS. An aspect of the invention is a pharmaceutical formulation comprising a pharmaceutically acceptable carrier, abiraterone acetate and particles consisting of a solid dispersion, said solid dispersion consisting of ARN-509 and HPMCAS. In an aspect, the weight-by-weight ratio of ARN-509:HPMCAS is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles comprising a solid dispersion, said solid dispersion comprising ARN-509 and HPMCAS LG. An aspect of the invention is a pharmaceutical formulation comprising a pharmaceutically acceptable carrier, abiraterone acetate and particles comprising a solid dispersion, said solid dispersion consisting of ARN-509 and HPMCAS LG. In an aspect, the weight-by-weight ratio of ARN-509:HPMCAS LG is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles comprising a solid dispersion, said solid dispersion comprising ARN-509 and HPMCAS LF. An aspect of the invention is a pharmaceutical formulation comprising a pharmaceutically acceptable carrier, abiraterone acetate and particles comprising a solid dispersion, said solid dispersion consisting of ARN-509 and HPMCAS LF. In an aspect, the weight-by-weight ratio of ARN-509:HPMCAS LF is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles consisting of a solid dispersion, said solid dispersion comprising ARN-509 and HPMCAS LG. An aspect of the invention is a pharmaceutical formulation comprising a pharmaceutically acceptable carrier, abiraterone acetate and particles consisting of a solid dispersion, said solid dispersion consisting of ARN-509 and HPMCAS LG. In an aspect, the weight-by-weight ratio of ARN-509:HPMCAS LG is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles consisting of a solid dispersion, said solid dispersion comprising ARN-509 and HPMCAS LF. An aspect of the invention is a pharmaceutical formulation comprising a pharmaceutically acceptable carrier, abiraterone acetate and particles consisting of a solid dispersion, said solid dispersion consisting of ARN-509 and HPMCAS LF. In an aspect, the weight-by-weight ratio of ARN-509:HPMCAS LF is 1:1, 1:2 or 1:3. In an aspect, the particles are obtainable, in particular are obtained, by spray drying as described herein.

In an aspect, the particles are obtainable, in particular are obtained, by melt extrusion as described herein.

An aspect of the invention is a pharmaceutical formulation comprising a pharmaceutically acceptable carrier, abiraterone acetate and a solid dispersion, said solid dispersion comprising ARN-509 and a poly(meth)acrylate copolymer. An aspect of the invention is a pharmaceutical formulation comprising a pharmaceutically acceptable carrier, abiraterone acetate and a solid dispersion, said solid dispersion consisting of ARN-509 and a poly(meth)acrylate copolymer. In an aspect, the weight-by-weight ratio of ARN-509:poly(meth)acrylate copolymer is 1:1, 1:2 or 1:3.

An aspect of the invention is a pharmaceutical formulation comprising a pharmaceutically acceptable carrier, abiraterone acetate and a solid dispersion, said solid dispersion comprising ARN-509 and Eudragit® L 100-55. An aspect of the invention is a pharmaceutical formulation comprising a pharmaceutically acceptable carrier, abiraterone acetate and a solid dispersion, said solid dispersion consisting of ARN-509 and Eudragit® L 100-55. In an aspect, the weight-by-weight ratio of ARN-509:Eudragit® L 100-55 is 1:1, 1:2 or 1:3.

An aspect of the invention is a pharmaceutical formulation comprising a pharmaceutically acceptable carrier, abiraterone acetate and a solid dispersion, said solid dispersion comprising ARN-509 and Eudragit® E 100. An aspect of the invention is a pharmaceutical formulation comprising a pharmaceutically acceptable carrier, abiraterone acetate and a solid dispersion, said solid dispersion consisting of ARN-509 and Eudragit® E 100. In an aspect, the weight-by-weight ratio of ARN-509:Eudragit® E 100 is 1:1, 1:2 or 1:3.

An aspect of the invention is a pharmaceutical formulation comprising a pharmaceutically acceptable carrier, abiraterone acetate and particles comprising a solid dispersion, said solid dispersion comprising ARN-509 and a poly(meth)acrylate copolymer. An aspect of the invention is a pharmaceutical formulation comprising a pharmaceutically acceptable carrier, abiraterone acetate and particles comprising a solid dispersion, said solid dispersion consisting of ARN-509 and a poly(meth)acrylate copolymer. In an aspect, the weight-by-weight ratio of ARN-509:poly(meth)acrylate copolymer is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles consisting of a solid dispersion, said solid dispersion comprising ARN-509 and a poly(meth)acrylate copolymer. An aspect of the invention is a pharmaceutical formulation comprising a pharmaceutically acceptable carrier, abiraterone acetate and particles consisting of a solid dispersion, said solid dispersion consisting of ARN-509 and a poly(meth)acrylate copolymer. In an aspect, the weight-by-weight ratio of ARN-509:poly(meth)acrylate copolymer is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles comprising a solid dispersion, said solid dispersion comprising ARN-509 and Eudragit® L 100-55. An aspect of the invention is a pharmaceutical formulation comprising a pharmaceutically acceptable carrier, abiraterone acetate and particles comprising a solid dispersion, said solid dispersion consisting of ARN-509 and Eudragit® L 100-55. In an aspect, the weight-by-weight ratio of ARN-509:Eudragit® L 100-55 is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles comprising a solid dispersion, said solid dispersion comprising ARN-509 and Eudragit® E 100. An aspect of the invention is a pharmaceutical formulation comprising a pharmaceutically acceptable carrier, abiraterone acetate and particles comprising a solid dispersion, said solid dispersion consisting of ARN-509 and Eudragit® E 100. In an aspect, the weight-by-weight ratio of ARN-509:Eudragit® E 100 is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles consisting of a solid dispersion, said solid dispersion comprising ARN-509 and Eudragit® L 100-55. An aspect of the invention is a pharmaceutical formulation comprising a pharmaceutically acceptable carrier, abiraterone acetate and particles consisting of a solid dispersion, said solid dispersion consisting of ARN-509 and Eudragit® L 100-55. In an aspect, the weight-by-weight ratio of ARN-509:Eudragit® L 100-55 is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and particles consisting of a solid dispersion, said solid dispersion comprising ARN-509 and Eudragit® E 100. An aspect of the invention is a pharmaceutical formulation comprising a pharmaceutically acceptable carrier, abiraterone acetate and particles consisting of a solid dispersion, said solid dispersion consisting of ARN-509 and Eudragit® E 100. In an aspect, the weight-by-weight ratio of ARN-509:Eudragit® E 100 is 1:1, 1:2 or 1:3. In an aspect, the particles are obtainable, in particular are obtained, by spray drying as described herein. In an aspect, the particles are obtainable, in particular are obtained, by melt extrusion as described herein.

An aspect of the invention is a pharmaceutical formulation comprising a pharmaceutically acceptable carrier, abiraterone acetate and a solid dispersion, said solid dispersion comprising ARN-509, a poly(meth)acrylate copolymer and HPMCAS. An aspect of the invention is a pharmaceutical formulation comprising a pharmaceutically acceptable carrier, abiraterone acetate and a solid dispersion, said solid dispersion consisting of ARN-509, a poly(meth)acrylate copolymer and HPMCAS. In an aspect, the weight-by-weight ratio of ARN-509:(poly(meth)acrylate copolymer and HPMCAS) is 1:1, 1:2 or 1:3. In an aspect, the weight-by-weight ratio of poly(meth)acrylate copolymer:HPMCAS ranges from 25:75 to 75:25, or is 25:75, 50:50, or 75:25; 50:50 being preferred.

An aspect of the invention is a pharmaceutical formulation comprising a pharmaceutically acceptable carrier, abiraterone acetate and a solid dispersion, said solid dispersion comprising ARN-509, Eudragit® L 100-55 and HPMCAS LG. An aspect of the invention is a pharmaceutical formulation comprising a pharmaceutically acceptable carrier, abiraterone acetate and a solid dispersion, said solid dispersion consisting of ARN-509, Eudragit® L 100-55 and HPMCAS LG. In an aspect, the weight-by-weight ratio of ARN-509:(Eudragit® L 100-55 and HPMCAS LG) is 1:1, 1:2 or 1:3. In an aspect, the weight-by-weight ratio of Eudragit® L 100-55:HPMCAS LG ranges from 25:75 to 75:25, or is 25:75, 50:50, or 75:25; 50:50 being preferred.

An aspect of the invention is a pharmaceutical formulation comprising a pharmaceutically acceptable carrier, abiraterone acetate and a solid dispersion, said solid dispersion comprising ARN-509, Eudragit® E 100 and HPMCAS LG. An aspect of the invention is a pharmaceutical formulation comprising a pharmaceutically acceptable carrier, abiraterone acetate and a solid dispersion, said solid dispersion consisting of ARN-509, Eudragit® E 100 and HPMCAS LG. In an aspect, the weight-by-weight ratio of ARN-509:(Eudragit® E 100 and HPMCAS LG) is 1:1, 1:2 or 1:3. In an aspect, the weight-by-weight ratio of Eudragit® E 100:HPMCAS LG ranges from 25:75 to 75:25, or is 25:75, 50:50, or 75:25; 50:50 being preferred.

An aspect of the invention is a pharmaceutical formulation comprising a pharmaceutically acceptable carrier, abiraterone acetate and a solid dispersion, said solid dispersion comprising ARN-509, Eudragit® L 100-55 and HPMCAS LF. An aspect of the invention is a pharmaceutical formulation comprising a pharmaceutically acceptable carrier, abiraterone acetate and a solid dispersion, said solid dispersion consisting of ARN-509, Eudragit® L 100-55 and HPMCAS LF. In an aspect, the weight-by-weight ratio of ARN-509:(Eudragit® L 100-55 and HPMCAS LF) is 1:1, 1:2 or 1:3. In an aspect, the weight-by-weight ratio of Eudragit® L 100-55:HPMCAS LF ranges from 25:75 to 75:25, or is 25:75, 50:50, or 75:25; 50:50 being preferred.

An aspect of the invention is a pharmaceutical formulation comprising a pharmaceutically acceptable carrier, abiraterone acetate and a solid dispersion, said solid dispersion comprising ARN-509, Eudragit® E 100 and HPMCAS LF. An aspect of the invention is a pharmaceutical formulation comprising a pharmaceutically acceptable carrier, abiraterone acetate and a solid dispersion, said solid dispersion consisting of ARN-509, Eudragit® E 100 and HPMCAS LF. In an aspect, the weight-by-weight ratio of ARN-509:(Eudragit® E 100 and HPMCAS LF) is 1:1, 1:2 or 1:3. In an aspect, the weight-by-weight ratio of Eudragit® E 100:HPMCAS LF ranges from 25:75 to 75:25, or is 25:75, 50:50, or 75:25; 50:50 being preferred.

An aspect of the invention is a pharmaceutical formulation comprising a pharmaceutically acceptable carrier, abiraterone acetate and particles comprising a solid dispersion, said solid dispersion comprising ARN-509, a poly(meth)acrylate copolymer and HPMCAS. An aspect of the invention is a pharmaceutical formulation comprising a pharmaceutically acceptable carrier, abiraterone acetate and particles comprising a solid dispersion, said solid dispersion consisting of ARN-509, a poly(meth)acrylate copolymer and HPMCAS. In an aspect, the weight-by-weight ratio of ARN-509:(poly(meth)acrylate copolymer and HPMCAS) is 1:1, 1:2 or 1:3. In an aspect, the weight-by-weight ratio of poly(meth)acrylate copolymer:HPMCAS ranges from 25:75 to 75:25, or is 25:75, 50:50, or 75:25; 50:50 being preferred. In an aspect, the particles are obtainable, in particular are obtained, by spray drying as described herein. In an aspect, the particles are obtainable, in particular are obtained, by melt extrusion as described herein. In an aspect, the poly(meth)acrylate copolymer is selected from Eudragit® L 100-55 and Eudragit® E 100. In an aspect the HPMCAS is selected from HPMCAS LG and HPMCAS LF, in particular the HPMCAS is HPMCAS LG.

An aspect of the invention is a pharmaceutical formulation comprising a pharmaceutically acceptable carrier, abiraterone acetate and particles consisting of a solid dispersion, said solid dispersion comprising ARN-509, a poly(meth)acrylate copolymer and HPMCAS. An aspect of the invention is a pharmaceutical formulation comprising a pharmaceutically acceptable carrier, abiraterone acetate and particles consisting of a solid dispersion, said solid dispersion consisting of ARN-509, a poly(meth)acrylate copolymer and HPMCAS. In an aspect, the weight-by-weight ratio of ARN-509:(poly(meth)acrylate copolymer and HPMCAS) is 1:1, 1:2 or 1:3. In an aspect, the weight-by-weight ratio of poly(meth)acrylate copolymer:HPMCAS ranges from 25:75 to 75:25, or is 25:75, 50:50, or 75:25; 50:50 being preferred. In an aspect, the particles are obtainable, in particular are obtained, by spray drying as described herein. In an aspect, the particles are obtainable, in particular are obtained, by melt extrusion as described herein. In an aspect, the poly(meth)acrylate copolymer is selected from Eudragit® L 100-55 and Eudragit® E 100. In an aspect the HPMCAS is selected from HPMCAS LG and HPMCAS LF, in particular the HPMCAS is HPMCAS LG.

In an aspect of the invention, the particles as described herein are obtainable, in particular are obtained, by melt-extruding a mixture comprising ARN-509 and a polymer as described herein, in particular HPMCAS, and subsequently milling said melt-extruded mixture. In an aspect, the particles as described herein are obtainable, in particular are obtained, by melt-extruding a mixture consisting of ARN-509 and a polymer as described herein, in particular HPMCAS, and subsequently milling said melt-extruded mixture. In an aspect, the weight-by-weight ratio of ARN-509:polymer as described herein, in particular HPMCAS, is 1:1, 1:2 or 1:3.

In an aspect of the invention, the particles as described herein are obtainable, in particular are obtained, by spray drying a mixture comprising ARN-509 and a polymer as described herein, in particular HPMCAS, in a suitable solvent. In an aspect, the particles as described herein are obtainable, in particular are obtained, by spray drying a mixture consisting of ARN-509 and a polymer as described herein, in particular HPMCAS, in a suitable solvent. In an aspect, the weight-by-weight ratio of ARN-509:polymer as described herein, in particular HPMCAS, is 1:1, 1:2 or 1:3.

An aspect of the invention is a pharmaceutical formulation comprising a pharmaceutically acceptable carrier, abiraterone acetate and a solid dispersion, said solid dispersion comprising ARN-509 and a polymer selected from HPMCAS, a poly(meth)acrylate copolymer, and mixtures thereof wherein the weight-by-weight ratio of ARN-509:polymer in the solid dispersion is 1:1.

An aspect of the invention is a pharmaceutical formulation comprising a pharmaceutically acceptable carrier, abiraterone acetate and a solid dispersion, said solid dispersion comprising ARN-509 and a polymer selected from HPMCAS, a poly(meth)acrylate copolymer, and mixtures thereof wherein the weight-by-weight ratio of ARN-509:polymer in the solid dispersion is 1:2.

An aspect of the invention is a pharmaceutical formulation comprising a pharmaceutically acceptable carrier, abiraterone acetate and a solid dispersion, said solid dispersion comprising ARN-509 and a polymer selected from HPMCAS, a poly(meth)acrylate copolymer, and mixtures thereof wherein the weight-by-weight ratio of ARN-509:polymer in the solid dispersion is 1:3.

In an aspect of the invention, the solid dispersion comprised in the pharmaceutical formulation as described herein does not contain a surfactant.

In an aspect of the invention, the particles comprising or consisting of a solid dispersion, said solid dispersion comprising or consisting of ARN 509 and a polymer selected from HPMCAS, a poly(meth)acrylate copolymer, and mixtures thereof, said particles being comprised in the pharmaceutical formulations as described herein do not contain a surfactant.

An aspect of the invention is a pharmaceutical formulation as described herein wherein the solid dispersion contains as the only active pharmaceutical ingredient ARN-509.

In the solid dispersions or particles or pharmaceutical formulations as described herein ARN-509 is present in base form or as a pharmaceutically acceptable addition salt, in particular as a pharmaceutically acceptable acid addition salt. Preferably, ARN-509 is present in base form.

In the granulates or pharmaceutical formulations as described herein abiraterone acetate is present in base form or as a pharmaceutically acceptable addition salt, in particular as a pharmaceutically acceptable acid addition salt. Preferably, abiraterone acetate is present in base form.

The pharmaceutically acceptable addition salts are meant to comprise the therapeutically active non-toxic salt forms. The acid addition salt forms can be obtained by treating the base form of ARN-509 or abiraterone acetate with an appropriate acid, such as inorganic acids, including but not limited to, hydrohalic acids, e.g. hydrochloric acid, hydrobromic acid and the like acids; sulfuric acid; nitric acid; phosphoric acid; metaphosphoric acid and the like acids; or organic acids, including but not limited to, acetic acid, trifluoroacetic acid, trimethylacetic acid, propanoic acid, hydroxyacetic acid, 2-hydroxypropanoic acid, 2-oxopropanoic acid, glycolic acid, oxalic acid, malonic acid, succinic acid, maleic acid, fumaric acid, malic acid, mandelic acid, tartaric acid, 2-hydroxy-1,2,3-propanetricarboxylic acid, methanesulfonic acid, ethanesulfonic acid, 1,2-ethanedisulfonic acid, 2-hydroxyethanesulfonic acid, benzoic acid, cinnamic acid, hydrocinnamic acid, benzenesulfonic acid, 4-methylbenzene-sulfonic acid, 2-naphthalenesulfonic acid, cyclohexanesulfamic acid, 2-hydroxybenzoic acid, 4-amino-2-hydroxybenzoic acid, hexanoic acid, cyclopentanepropionic acid, 3-(4-hydroxybenzoyl)benzoic acid, 4-methylbicyclo-[2.2.2]oct-2-ene-1-carboxylic acid, glucoheptonic acid, tertiary butylacetic acid, lauryl sulfuric acid, gluconic acid, glutamic acid, hydroxynaphthoic acid, stearic acid, muconic acid, butyric acid, phenylacetic acid, phenylbutyric acid, valproic acid, and the like acids.

Conversely said salt forms can be converted by treatment with an appropriate base into the free base form.

Also included are the hydrates, the solvent addition forms and mixtures thereof which ARN-509 and its salts are able to form. Examples of such forms are e.g. hydrates, alcoholates and the like, for instance an ethanolate.

In general, doses of ARN-509 employed for adult human treatment are typically in the range from 0.01 mg to 5000 mg per day. In one aspect, doses employed for adult human treatment are from about 1 mg to about 1000 mg per day. In another aspect, doses employed for adult human treatment are from about 100 mg to about 500 mg per day. In another aspect, the dose employed for ARN-509 for adult human treatment is 240 mg per day. The exact dosage and frequency of administration of ARN-509 may depend on the particular condition being treated, the severity of the condition being treated, the age, weight and general physical condition of the particular patient as well as other medication the individual may be taking, as is known to those skilled in the art.

Furthermore, it is evident that said daily amounts may be lowered or increased depending on the response of the treated subject and/or depending on the evaluation of the physician prescribing ARN-509. In another aspect, the dose employed for abiraterone acetate for adult human treatment currently is 1000 mg per day. The exact dosage and frequency of administration of abiraterone acetate may depend on the particular condition being treated, the severity of the condition being treated, the age, weight and general physical condition of the particular patient as well as other medication the individual may be taking, as is known to those skilled in the art. Furthermore, it is evident that said daily amounts may be lowered or increased depending on the response of the treated subject and/or depending on the evaluation of the physician prescribing abiraterone acetate. The doses mentioned herein are therefore only a guideline and are not intended to limit the scope or use of the invention to any extent. The dose of abiraterone acetate employed for adult human treatment may therefore range from 500 mg to 5000 mg per day, 100 mg to 1000 mg per day or 1 mg to 1000 mg per day. In an aspect of the invention, the daily dose is conveniently presented in a single dose or in divided doses administered simultaneously (or over a short period of time) or at appropriate intervals, for example as two, three, four or more sub-doses per day. In an aspect of the invention, the daily dose is administered in 4 divided doses. In an aspect of the invention, the daily dose is administered in 4 divided doses administered simultaneously (or over a short period of time). In an aspect of the invention, the daily dose is administered in 3 divided doses. In an aspect of the invention, the daily dose is administered in 3 divided doses administered simultaneously (or over a short period of time). In an aspect of the invention, the daily dose is administered in 2 divided doses. In an aspect of the invention, the daily dose is administered in 2 divided doses administered simultaneously (or over a short period of time). In an aspect of the invention, the daily dose is administered in 1 dose. In an aspect of the invention, the daily dose is administered in 1 dose administered simultaneously (or over a short period of time).

In an aspect of the invention, the pharmaceutical formulation comprises 240 mg of ARN-509 and 1000 mg of abiraterone acetate.

In an aspect of the invention, the pharmaceutical formulation comprises 120 mg of ARN-509 and 500 mg of abiraterone acetate.

In an aspect of the invention, the pharmaceutical formulation comprises 80 mg of ARN-509 and 333.3 mg of abiraterone acetate.

In an aspect of the invention, the pharmaceutical formulation comprises 60 mg of ARN-509 and 250 mg of abiraterone acetate.

In an aspect of the invention, the pharmaceutical formulation comprises 240 mg of ARN-509 and 1000 mg of abiraterone acetate. The pharmaceutical formulation is administered once daily.

In an aspect of the invention, the pharmaceutical formulation comprises 120 mg of ARN-509 and 500 mg of abiraterone acetate. Two of said formulations are administered daily, preferably simultaneously (or over a short period of time).

In an aspect of the invention, the pharmaceutical formulation comprises 80 mg of ARN-509 and 333.3 mg of abiraterone acetate. Three of said formulations are administered daily, preferably simultaneously (or over a short period of time).

In an aspect of the invention, the pharmaceutical formulation comprises 60 mg of ARN-509 and 250 mg of abiraterone acetate. Four of said formulations are administered daily, preferably simultaneously (or over a short period of time).

With the pharmaceutical formulations of the present invention which are co-formulated compositions comprising ARN-509 and abiraterone acetate, the pill burden for the patient, in particular the cancer patient, can be reduced and hence therapy adherence and therapy efficiency can be improved.

The formulations of the present invention can also be used in combination with another anticancer agent, in particular with another anti prostate cancer agent. The formulations of the present invention can be combined with a glucocorticoid selected from the group consisting of prednisone, prednisolone, methylprednisolone, dexamethasone and pharmaceutically acceptable salts and acetates thereof. In an aspect of the present invention the pharmaceutical formulation of the present invention can be combined with a glucocorticoid selected from the group consisting of prednisone, prednisolone acetate, methylprednisolone acetate, methylprednisolone, prednisolone sodium phosphate, prednisolone phosphate, prednisolone sodium succinate, methylprednisolone sodium succinate, dexamethasone sodium phosphate, and dexamethasone acetate. In an aspect of the invention, the pharmaceutical formulations of the invention can be combined with hydrocortisone, hydrocortisone acetate, hydrocortisone sodium succinate, hydrocortisone cypionate, hydrocortisone butyrate, hydrocortisone valerate, hydrocortisone probutate, or corticotropin. In an aspect of the invention, the pharmaceutical formulations of the present invention can be combined with prednisone. In an aspect of the invention, the pharmaceutical formulations of the present invention can be combined with prednisolone.

Thus, the present invention also relates to a combination of a pharmaceutical formulation according to the invention and another anticancer agent.

The present invention also relates to a combination of a pharmaceutical formulation according to the invention and a glucocorticoid selected from the group consisting of prednisone, prednisolone, methylprednisolone, dexamethasone and pharmaceutically acceptable salts and acetates thereof. The present invention also relates to a combination of a pharmaceutical formulation according to the invention and a glucocorticoid selected from the group consisting of prednisone, prednisolone acetate, methylprednisolone acetate, methylprednisolone, prednisolone sodium phosphate, prednisolone phosphate, prednisolone sodium succinate, methylprednisolone sodium succinate, dexamethasone sodium phosphate, and dexamethasone acetate. The present invention also relates to a combination of a pharmaceutical formulation according to the invention and hydrocortisone, hydrocortisone acetate, hydrocortisone sodium succinate, hydrocortisone cypionate, hydrocortisone butyrate, hydrocortisone valerate, hydrocortisone probutate, or corticotropin. The present invention also relates to a combination of a pharmaceutical formulation according to the invention and prednisone. The present invention also relates to a combination of a pharmaceutical formulation according to the invention and prednisolone.

The term “solid dispersion” as described herein means a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermo-dynamics, such a solid dispersion will be called “a solid solution” herein. Solid solutions are preferred physical systems because the components therein are usually readily bioavailable to the organisms to which they are administered. This advantage can probably be explained by the ease with which said solid solutions can form liquid solutions when contacted with a liquid medium such as gastric juice. The ease of dissolution may be attributed at least in part to the fact that the energy required for dissolution of the components from a solid solution is less than that required for the dissolution of components from a crystalline or microcrystalline solid phase.

The term “solid dispersion” also comprises dispersions which are less homogenous throughout than solid solutions. Such dispersions are not chemically and physically uniform throughout or comprise more than one phase. For example, the term “solid dispersion” also relates to a system in a solid state comprising at least two components (a) and (b) and having domains or small regions wherein amorphous, microcrystalline or crystalline (a), or amorphous, microcrystalline or crystalline (b), or both, are dispersed more or less evenly in another phase comprising (b), or (a), or a solid solution comprising (a) and (b). Said domains are regions distinctively marked by some physical feature, small in size compared to the size of the system as a whole, and evenly and randomly distributed throughout the system.

Preferred are solid dispersions or particles comprising or consisting of a solid dispersion as described herein wherein ARN-509 is in a non-crystalline phase as these have an intrinsically faster dissolution rate than those wherein part or all of ARN-509 is in a microcrystalline or crystalline form.

Alternatively, the solid dispersions may be in the form of a dispersion wherein amorphous or microcrystalline ARN-509 or amorphous or microcrystalline polymer selected from HPMCAS, a poly(meth)acrylate copolymer, and mixtures thereof is dispersed more or less evenly in a solid solution comprising ARN-509 and polymer selected from HPMCAS, a poly(meth)acrylate copolymer, and mixtures thereof.

In an aspect of the invention ARN-509 is present in the solid dispersions as described herein in amorphous form.

In an aspect of the invention the solid dispersion as described herein is a solid solution.

In an aspect of the invention, the pharmaceutical formulations as described herein comprise amorphous ARN-509.

In an aspect of the invention, the pharmaceutical formulations as described herein comprise crystalline abiraterone acetate.

In an aspect of the invention, the pharmaceutical formulations as described herein comprise amorphous ARN-509 and crystalline abiraterone acetate.

In an aspect of the invention, the pharmaceutical formulations as described herein comprise amorphous ARN-509 and granulates comprising crystalline abiraterone acetate.

Various techniques exist for preparing the solid dispersions of the invention including melt-extrusion (e.g. hot melt extrusion), spray-drying and solution-evaporation, in particular hot melt-extrusion and spray-drying, spray-drying being preferred.

The particles according to the invention can be prepared by first preparing a solid dispersion of the components, and then optionally grinding or milling said dispersion.

The melt-extrusion process comprises the following steps:
a) mixing ARN-509 and a polymer selected from HPMCAS, a poly(meth)acrylate copolymer, and mixtures thereof,
b) optionally blending additives with the thus obtained mixture,
c) heating the thus obtained blend until one obtains a homogenous melt,
d) forcing the thus obtained melt through one or more nozzles; and
e) cooling the melt till it solidifies.

The terms “melt” and “melting” do not only mean the alteration from a solid state to a liquid state, but can also refer to a transition to a glassy state or a rubbery state, and in which it is possible for one component of the mixture to get embedded more or less homogeneously into the other. In particular cases, one component will melt and the other component(s) will dissolve in the melt thus forming a solution, which upon cooling may form a solid solution having advantageous dissolution properties.

One important parameter of melt extrusion is the temperature at which the melt-extruder is operating. For the melt extrusion process of the present invention, the operating temperature preferably ranges between about 160° C. and about 190° C., more preferably ranges between about 160° C. and 175° C. The lower temperature limit is defined by the point at which ARN-509 is still melting during extrusion with a given set of extrusion conditions. When ARN-509 is not completely molten, the extrudate may not provide the desired bioavailability. When the viscosity of the mixture is too high, the process of melt extrusion will be difficult. At higher temperatures the components may decompose to an unacceptable level. A person skilled in the art will recognize the most appropriate temperature range to be used.

The throughput rate is also of importance because the components may start to decompose when they remain too long in contact with the heating element.

It will be appreciated that the person skilled in the art will be able to optimize the parameters of the melt extrusion process within the above given ranges. The working temperatures will also be determined by the kind of extruder or the kind of configuration within the extruder that is used. Most of the energy needed to melt, mix and dissolve the components in the extruder can be provided by the heating elements. However, the friction of the material within the extruder may also provide a substantial amount of energy to the mixture and aid in the formation of a homogenous melt of the components.

A person skilled in the art will recognize the most appropriate extruder, such as, for example, a single screw, a twin screw extruder or a multi-screw extruder, for the preparation of the subject-matter of the present invention.

Spray-drying of a mixture of the components in a suitable solvent also yields a solid dispersion of said components or particles comprising or consisting of a solid dispersion of said components and may be a useful alternative to the melt-extrusion process, particularly in those cases where the polymer selected from HPMCAS, a poly(meth)acrylate copolymer, and mixtures thereof is not sufficiently stable to withstand the extrusion conditions and where residual solvent can effectively be removed from the solid dispersion. Yet another possible preparation consists of preparing a mixture of the components in a suitable solvent, pouring said mixture onto a large surface so as to form a thin film, and evaporating the solvent therefrom.

Solvents suitable for spray-drying can be any organic solvent in which ARN-509 and a polymer selected from HPMCAS, a poly(meth)acrylate copolymer, and mixtures thereof, in particular HPMCAS LG or HPMCAS LF or Eudragit® L 100-55 and Eudragit® E 100, are miscable. In an aspect of the invention, the boiling point of the solvent is lower than the Tg (glass transition temperature) of the solid dispersion. In addition, the solvent should have relatively low toxicity and be removed from the dispersion to a level that is acceptable according to The International Committee on Harmonization (ICH) guidelines. Removal of solvent to this level may require a post drying step such as for instance tray-drying, subsequent to the spray-drying process. Solvents include alcohols such as methanol, ethanol, n-propanol, iso-propanol, and butanol, in particular methanol; ketones such as acetone, methyl ethyl ketone and methyl iso-butyl ketone; esters such as ethyl acetate and propylacetate; and various other solvents such as acetonitrile, dichloromethane, toluene, and 1,1,1-trichloroethane. Lower volatility solvents such as dimethyl acetamide or dimethylsulfoxide can also be used. In an aspect of the invention, the solvent suitable for spray drying is a mixture of solvents. In an aspect of the invention the solvent for spray drying is a mixture of an alcohol and dichloromethane, in particular a mixture of methanol and dichloromethane, more in particular a mixture of methanol and dichloromethane 6:4 (w:w) or 5:5 (w/w), 6:4 (w:w) being preferred.

The particles of the solid dispersion of ARN-509 and a polymer selected from HPMCAS, a poly(meth)acrylate copolymer, and mixtures thereof, as described herein have a d⁵⁰ of about 1500 μm, of about 1000 μm, of about 500 μm, of about 400 μm, of about 250 μm, of about 200 μm, of about 150 μm, of about 125 μm, of about 100 μm, of about 70 μm, of about 65 μm, of about 60 μm, of about 55 μm, of about 50 μm, of about 45 μm, of about 40 μm, of about 35 μm, of about 30 μm, of about 25 μm, or of about 20 μm. Particles obtained by spray drying have preferably a d⁵⁰-value falling in the range from about 20 μm to about 100 μm, in particular a d⁵⁰-value falling in the range from about 20 μm to about 70 μm, more in particular a d⁵⁰-value falling in the range from about 40 μm to about 50 μm, more in particular a d⁵⁰-value of about 20 μm, of about 25 μm, of about 30 μm, of about 35 μm, of about 40 μm, of about 45 μm, of about 50 μm, of about 55 μm, of about 60 μm, of about 65 μm, or of about 70 μm.

As used herein, the term d⁵⁰ has its conventional meaning as known to the person skilled in the art and can be measured by art-known particle size measuring techniques such as, for example, sedimentation field flow fractionation, photon correlation spectroscopy, laser diffraction or disk centrifugation. The d⁵⁰ mentioned herein may be related to volume distributions of the particles. In that instance, by “a d⁵⁰ of 50 μm” it is meant that at least 50% of the volume of the particles has a particle size of less than 50 μm. The same applies to the other particle sizes mentioned. In a similar manner, the d⁵⁰ particle size may be related to weight distributions of the particles. In that instance, by “d⁵⁰ of 50 μm” it is meant that at least 50% of the weight of the particles has a particle size of less than 50 μm. The same applies to the other particle sizes mentioned. Usually volume and weight distribution result in the same or about the same value for the average particle size.

The particle size can be an important factor determining the tabletting speed, in particular the flowability and therefore the manufacturability on a large scale of a particular dosage form or formulation, and the quality of the final product. Particle size is also an important factor for tablet strength, compactability. For instance, for capsules, the particle size may range preferably from about 100 to about 1500 μm (d⁵⁰); for tablets the particle size is preferably less than 250 μm, more preferably less than 100 μm (d⁵⁰). Too small particles (<10-20 μm) often cause sticking on the tablet punches and manufacturability issues.

The particles or solid dispersions as described herein may further comprise one or more pharmaceutically acceptable excipients such as, for example, plasticizers, flavors, colorants, preservatives and the like. Especially in case of preparation by hot melt extrusion, said excipients should not be heat-sensitive, in other words, they should not show any appreciable degradation or decomposition at the working temperature of the melt-extruder. In an aspect of the invention, the particles or solid dispersions as described herein do not comprise one or more pharmaceutically acceptable excipients, but the the particles or solid dispersions consist of ARN-509 and a polymer selected from HPMCAS, a poly(meth)acrylate copolymer, and mixtures thereof.

Suitable plasticizers are pharmaceutically acceptable and include low molecular weight polyalcohols such as ethylene glycol, propylene glycol, 1,2 butylene glycol, 2,3-butylene glycol, styrene glycol; polyethylene glycols such as diethylene glycol, triethylene glycol, tetraethylene glycol; other polyethylene glycols having a molecular weight lower than 1,000 g/mol; polypropylene glycols having a molecular weight lower than 200 g/mol; glycol ethers such as monopropylene glycol monoisopropyl ether; propylene glycol monoethyl ether; diethylene glycol monoethyl ether; ester type plasticizers such as triethyl citrate, sorbitol lactate, ethyl lactate, butyl lactate, ethyl glycolate, allyl glycollate; and amines such as monoethanolamine, diethanolamine, triethanolamine, monoisopropanolamine; triethylenetetramine, 2-amino-2-methyl-1,3-propanediol and the like. Of these, the low molecular weight polyethylene glycols, ethylene glycol, low molecular weight polypropylene glycols and especially propylene glycol are preferred.

In an aspect of the invention, the particles or solid dispersions as described herein do not contain a plasticizer.

At first instance, with the present invention pharmaceutical formulations for oral administration such as tablets and capsules are envisaged, but the pharmaceutical formulations of the present invention can also be used for rectal administration. Preferred formulations are those adapted for oral administration shaped as a tablet. They can be produced by conventional tabletting techniques with conventional ingredients or excipients (pharmaceutically acceptable carrier) and with conventional tabletting machines. Because of the good flow properties and an acceptable density of a blend of abiraterone acetate, in particular granulates of abiraterone acetate, and the solid dispersion of ARN-509, extra steps in the manufacturing process to improve flow properties or to improve density of the mixture, such as roller compaction, can be avoided while still obtaining good content uniformity of the active ingredients within the formulation and over a whole batch of formulations prepared. In order to facilitate the swallowing of such a formulation by a mammal, it is advantageous to give the formulations, in particular tablets, an appropriate shape. A film coat on the tablet may further contribute to the ease with which it can be swallowed. A preferred tablet of the present invention is an oblong shaped tablet, in particular an oblong shaped tablet with a length of ≤19 mm.

The formulations of the invention, in particular the tablets, include one or more conventional excipients (pharmaceutically acceptable carrier) such as disintegrants, diluents, fillers, binders, buffering agents, lubricants, glidants, thickening agents, sweetening agents, flavors, and colors. Some excipients can serve multiple purposes. In an aspect, the formulations of the present invention include a disintegrant, a diluent or filler, a lubricant and glidant. In an aspect, the formulations of the present invention include a disintegrant, a diluent or filler, a lubricant, glidant, a wetting agent and a binder. In an aspect, the formulations of the present invention include a disintegrant, a diluent or filler, a lubricant, glidant, a wetting agent and a binder, wherein the wetting agent or part of it and the binder are present in granulates of abiraterone acetate. The amount of wetting agent in the tablets or pharmaceutical formulations according to the present invention may conveniently range from about 0.5 to about 5% (w/w) and preferably range from about 0.5 to 3% (w/w) or from about 0.5 to 1.5% (w/w) or from about 0.5 to 1% (w/w).

Suitable disintegrants are those that have a large coefficient of expansion. Examples thereof are hydrophilic, insoluble or poorly water-soluble crosslinked polymers such as crospovidone (crosslinked polyvinylpyrrolidone) and croscarmellose sodium (crosslinked sodium carboxymethylcellulose). The amount of disintegrant in the tablets according to the present invention may conveniently range from about 3 to about 15% (w/w) and preferably range from about 3 to 7%, in particular is about 5 or 6% (w/w). Because disintegrants by their nature yield sustained release formulations when employed in bulk, it is advantageous to dilute them with an inert substance called a diluent or filler.

A variety of materials may be used as diluents or fillers. Examples are lactose monohydrate, anhydrous lactose, sucrose, dextrose, mannitol, sorbitol, starch, cellulose (e.g. micro-crystalline cellulose (Avicel™), silicified microcrystalline cellulose), dihydrated or anhydrous dibasic calcium phosphate, and others known in the art, and mixtures thereof (e.g. spray-dried mixture of lactose monohydrate (75%) with microcrystalline cellulose (25%) which is commercially available as Microcelac™). Preferred is microcrystalline cellulose, silicified microcrystalline cellulose or lactose monohydrate. The amount of diluent or filler in the tablets may conveniently range from about 20% to about 70% (w/w) and preferably ranges from about 55% to about 60% (w/w) or from about 30% to about 60% (w/w) or from about 30% to about 45% (w/w).

Lubricants and glidants can be employed in the manufacture of certain dosage forms, and will usually be employed when producing tablets. Examples of lubricants and glidants are hydrogenated vegetable oils, e.g hydrogenated Cottonseed oil, magnesium stearate, stearic acid, sodium lauryl sulfate, magnesium lauryl sulfate, colloidal silica, colloidal anhydrous silica, talc, mixtures thereof, and others known in the art.

Interesting lubricants are magnesium stearate, and mixtures of magnesium stearate with colloidal silica. A preferred lubricant is magnesium stearate. A preferred glidant is colloidal anhydrous silica.

Glidants generally comprise 0.2 to 7.0% of the total tablet weight, in particular 0.5 to 1.5%, more in particular 1 to 1.5% (w/w).

Lubricants generally comprise 0.2 to 7.0% of the total tablet weight, in particular 0.2 to 1%, more in particular 0.5 to 1% (w/w).

Other excipients such as coloring agents and pigments may also be added to the formulations of the invention. Coloring agents and pigments include titanium dioxide and dyes suitable for food. A coloring agent is an optional ingredient in the formulation of the invention, but when used the coloring agent can be present in an amount up to 3.5% based on the total tablet weight.

Flavors are optional in the formulation and may be chosen from synthetic flavor oils and flavoring aromatics or natural oils, extracts from plants leaves, flowers, fruits and so forth and combinations thereof. These may include cinnamon oil, oil of wintergreen, peppermint oils, bay oil, anise oil, eucalyptus, thyme oil. Also useful as flavors are vanilla, citrus oil, including lemon, orange, grape, lime and grapefruit, and fruit essences, including apple, banana, pear, peach, strawberry, raspberry, cherry, plum, pineapple, apricot and so forth, The amount of flavor may depend on a number of factors including the organoleptic effect desired. Generally the flavor will be present in an amount from about 0% to about 3% (w/w).

As known in the art, tablet blends may be dry-granulated or wet-granulated before tabletting. In an aspect of the invention the abiraterone acetate is wet-granulated in a fluid bed granulator, such as for example a GEA Sirocco 300 or a Niro Aeromatic D600, resulting in abiraterone granulates. In an aspect, the inlet temperature of the fluid bed may vary between 25° C. to 80° C. or between 25° C. to 90° C.; the outlet temperature may vary between 25° C. to 50° C. or between 25° C. to 80° C.; the air flow may range between 500 to 2200 m³/h or between 500 to 4500 m³/h; the solution flow rate may range from 170 to 4200 g/min or between 0.200 to 2 kg/min, the atomizing air pressure may range from 2-6 bar or between 1.00 to 5.00 bar. In an aspect, the abiraterone acetate is wet-granulated with a binder solution comprising a solvent, such as for example water, a binder, such as for example a polymer, e.g. hypromellose, and a wetting agent, such as for example sodium lauryl sulfate. In an aspect, prior to being granulated with a binder solution, the abiraterone acetate is mixed with a suitable diluent, such as for example lactose monohydrate, and a suitable disintegrant, such as for example croscarmellose sodium.

The tabletting process itself is otherwise standard and readily practised by forming a tablet from desired blend or mixture of ingredients into the appropriate shape using a conventional tablet press. In an aspect, the process of making the blend or mixture of ingredients does not contain a roller compaction step.

In an aspect, the present invention comprises a process for preparing a pharmaceutical formulation as described herein, comprising the steps of:
a) preparing a solid dispersion of ARN-509 and a polymer selected from HPMCAS, a poly(meth)acrylate copolymer, and mixtures thereof;
b) preparing a granulate comprising abiraterone acetate;
c) mixing the solid dispersion of a) and the granulate of b) and a pharmaceutically acceptable carrier.

The resulting blend can be compressed into tablets or filled in capsules.

Unexpectedly it was found that in the process for preparing a pharmaceutical formulation as described herein a flow property improving step or a density improving step, such as a roller compaction step, in particular roller compaction of the solid dispersion powder, can be avoided.

Tablets of the present invention may further be film-coated e.g. to improve taste, to provide ease of swallowing and an elegant appearance. Many suitable polymeric film-coating materials are known in the art. In an aspect, the film-coating material is Opadry II 85F210036 Green. Other suitable film-forming polymers also may be used herein, including, hydroxypropylcellulose, hydroxypropyl methylcellulose (HPMC), especially HPMC 2910 5 mPa·s, and acrylate-methacrylate copolymers. A preferred film-coating material is a water permeable film-coating material, such as for example the HPMC coating Opadry II 32F220009. Besides a film-forming polymer, the film coat may further comprise a plasticizer (e.g. propylene glycol) and optionally a pigment (e.g. titanium dioxide). The film-coating suspension may also contain talc as an anti-adhesive. In tablets according to the invention, the film coat in terms of weight preferably accounts for about 3% (w/w) or less of the total tablet weight.

Preferred formulations are those wherein the weight of the particles or solid dispersions as described herein ranges from 20 to 40%, in particular from 25 to 35% of the total weight of the formulation.

In an embodiment of the invention, the pharmaceutical formulations demonstrate bioequivalence for the abiraterone component with Zytiga® (single agent abiraterone acetate tablets) currently on the market. The pharmaceutical formulations provide plasma levels of abiraterone that are equivalent to the plasma levels of abiraterone obtained with Zytiga®.

In studies to determine bioequivalence after a single dose, the parameters to be analysed are AUC₍₀₋ₜ₎, or, when relevant, AUC₍₀₋₇₂ₕ₎, and Cₘₐₓ. For these parameters, the 90% confidence interval for the ratio of the test and reference products should be contained within the acceptance interval of 80.00-125.00%. To be inside the acceptance interval the lower bound should be ≥80.00% when rounded to two decimal places and the upper bound should be ≤125.00% when rounded to two decimal places. AUC₍₀₋ₜ₎ should preferably cover at least 80% of AUC_{(0-∞)}. Additional parameters that may be reported are AUC_{(0-∞)} and tₘₐₓ.

In the above,

AUC₍₀₋ₜ₎: Area under the plasma concentration curve from administration to last observed concentration at time t;

AUC_{(0-∞)}: Area under the plasma concentration curve extrapolated to infinite time;

AUC₍₀₋₇₂ₕ₎: Area under the plasma concentration curve from administration to 72 h;

Cₘₐₓ: Maximum plasma concentration;

tₘₐₓ: Time until Cₘₐₓ is reached.

In an embodiment of the invention, the pharmaceutical formulations demonstrate bioequivalence for the ARN-509 component with the single agent ARN-509 product currently in clinical study (phase 3). The pharmaceutical formulations provide plasma levels of ARN-509 that are equivalent to the plasma levels of ARN-509 obtained with the single agent product.

The present invention further concerns a process of preparing solid dispersions as described herein, comprising blending ARN-509 and a polymer as described herein, in particular HPMCAS, and extruding said blend at a temperature in the range from about 160° C. to about 190° C.

The present invention further concerns a process of preparing particles as described herein, comprising blending ARN-509 and a polymer as described herein, in particular HPMCAS, extruding said blend at a temperature in the range from about 160° C. to about 190° C., grinding the extrudate, and optionally sieving the particles.

Suitable extruders that may be used are the Haake mini-extruder, Leistritz 18 mm extruder, and the Leistritz 27 mm extruder.

The present invention further concerns a process of preparing particles or solid dispersions as described herein comprising mixing ARN-509 and a polymer as described herein, in particular HPMCAS, in a suitable solvent and spray drying said mixture. In an aspect, the suitable solvent is a mixture of dichloromethane and methanol. In an aspect, the suitable solvent is a mixture of dichloromethane and methanol wherein the weight:weight ratio of dichloromethane to methanol in the mixture is 4:6 or 5:5, 4:6 being preferred.

A preferred crystalline form of ARN-509 for preparing the solid dispersions or particles as described herein is Form B, which is an anhydrous crystalline form (see hereinafter and reference is also made to WO2013/184681, which is incorporated herein by reference).

The invention also relates to a method of treating an androgen receptor (AR)-related disease or condition, in particular cancer, more in particular prostate cancer, including but not limited to castration-resistant prostate cancer, metastatic castration resistant prostate cancer, chemotherapy-naive metastatic castration resistant prostate cancer, biochemically relapsed hormone sensitive prostate cancer, or high-risk, non-metastatic castration-resistant prostate cancer, in a mammal, in particular a human, which comprises administering, in particular orally, to said mammal, in particular human, an effective anticancer amount of a pharmaceutical formulation as described herein.

The invention further concerns the use of a pharmaceutical formulation as described herein, for the manufacture of a medicament for treating an androgen receptor (AR)-related disease or condition, in particular cancer, more in particular prostate cancer, including but not limited to castration-resistant prostate cancer, metastatic castration resistant prostate cancer, chemotherapy-naive metastatic castration resistant prostate cancer, biochemically relapsed hormone sensitive prostate cancer, or high-risk, non-metastatic castration-resistant prostate cancer. Or, alternatively, the invention concerns a pharmaceutical formulation as described herein for use in the treatment of an androgen receptor (AR)-related disease or condition, in particular cancer, more in particular prostate cancer, including but not limited to castration-resistant prostate cancer, metastatic castration resistant prostate cancer, chemotherapy-naive metastatic castration resistant prostate cancer, biochemically relapsed hormone sensitive prostate cancer, or high-risk, non-metastatic castration-resistant prostate cancer.

The invention also relates to a pharmaceutical package suitable for commercial sale comprising a container, a pharmaceutical formulation as described herein, and associated with said package written matter.

In an embodiment, the pharmaceutical formulations of the invention are packed in bottles, e.g. HDPE bottles, optionally flushed with nitrogen, or in blisters, optionally flushed with nitrogen.

The term “about” as used herein in connection with a numerical value is meant to have its usual meaning in the context of the numerical value. Where necessary the word “about” may be replaced by the numerical value ±10%, or ±5%, or ±2%, or ±1%. All documents cited herein are incorporated by reference in their entirety.

The following examples are intended to illustrate the present invention.

### Example 1: ARN-509 Forms

For the preparation of different (crystalline) forms of ARN-509, reference is made to WO2013/184681, which is incorporated herein by reference. Different (crystalline or amorphous) forms of ARN-509 can be used to prepare the solid dispersions, particles or formulations according to the present invention.

A preferred form of ARN-509 for use in the preparation of the solid dispersions, particles or formulations according to the present invention is ARN-509 Form B, which is an anhydrous crystal. It was prepared by suspending ARN-509 Form A (reference is made to WO2013/184681, including for the diffraction data) in USP water and heating the slurry to 55±5° C., holding at said temperature for at least 24 hours, followed by cooling the slurry to 25±5° C. The resulting slurry was filtered, and the wet cake washed once with USP water. The wet cake was unloaded from the filter and dried under vacuum to afford ARN-509 Form B. Reference is also made to Example 2 below.

Solubility of Form A: 0.01 mg/ml in water.

Solubility of Form B: 0.004 mg/ml in water.

### Example 2

Characterisation of ARN-509 Form B

Powder XRD

X-ray powder diffraction (XRPD) analyses were carried out on a PANalytical (Philips) X'PertPRO MPD diffractometer. The instrument is equipped with a Cu LFF X-ray tube.

The compound was spread on a zero background sample holder.

Instrument Parameters

generator voltage: 45 kV

generator amperage: 40 mA

geometry: Bragg-Brentano

stage: spinner stage

Measurement Conditions

scan mode: continuous

scan range: 3 to 50° 2θ

step size: 0.02°/step

counting time: 30 sec/step

spinner revolution time: 1 sec

radiation type: CuKα

Incident beam path

| | | |
|---|---|---|
| program. divergence slit: | 15 | mm |
| Soller slit: | 0.04 | rad |
| beam mask: | 15 | mm |

| | |
|---|---|
| anti scatter slit: | 1° |
| beam knife: | + |

Diffracted beam path

| long anti scatter shield: | + | |
|---|---|---|
| Soller slit: | 0.04 | rad |

| | |
|---|---|
| Ni filter: | + |
| detector: | X'Celerator |

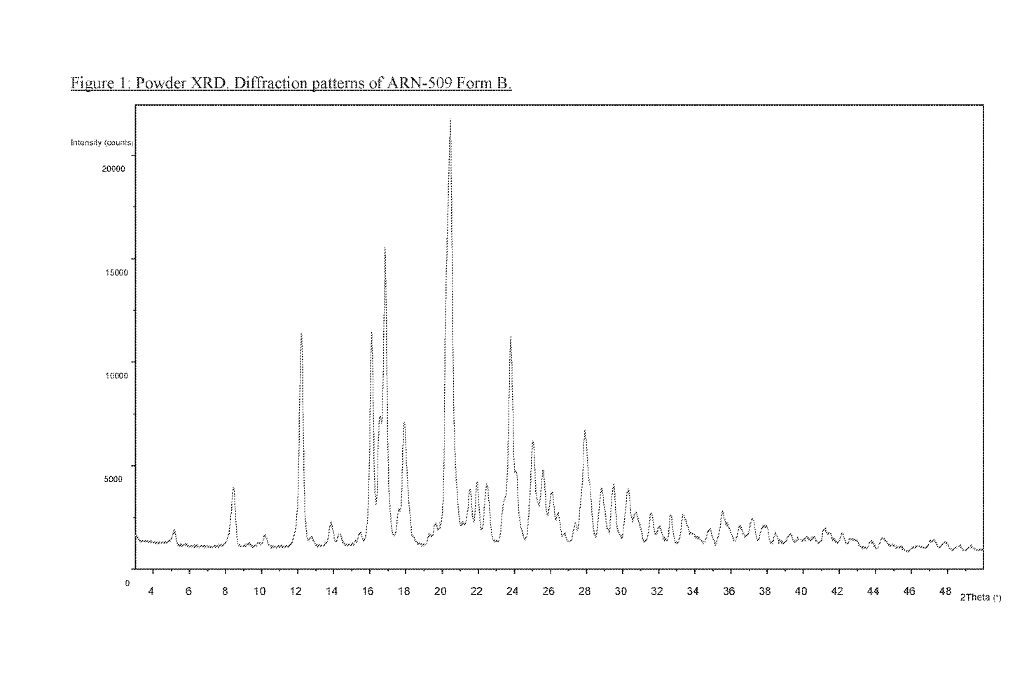

The X-ray powder diffraction pattern of ARN-509 Form B shows diffraction peaks without the presence of a halo, indicating that this compound is present as a crystalline product. The XRD pattern of ARN-509 Form B is shown in FIG. 1.

Infrared spectrometry (Micro ATR-IR)

The samples were analyzed using a suitable microATR accessory.

apparatus: Thermo Nexus 670 FTIR spectrometer

number of scans: 32

resolution: 1 cm⁻¹

wavelength range: 4000 to 400 cm⁻¹

detector: DTGS with KBr windows

beamsplitter: Ge on KBr

micro ATR accessory: Harrick Split Pea with Si crystal

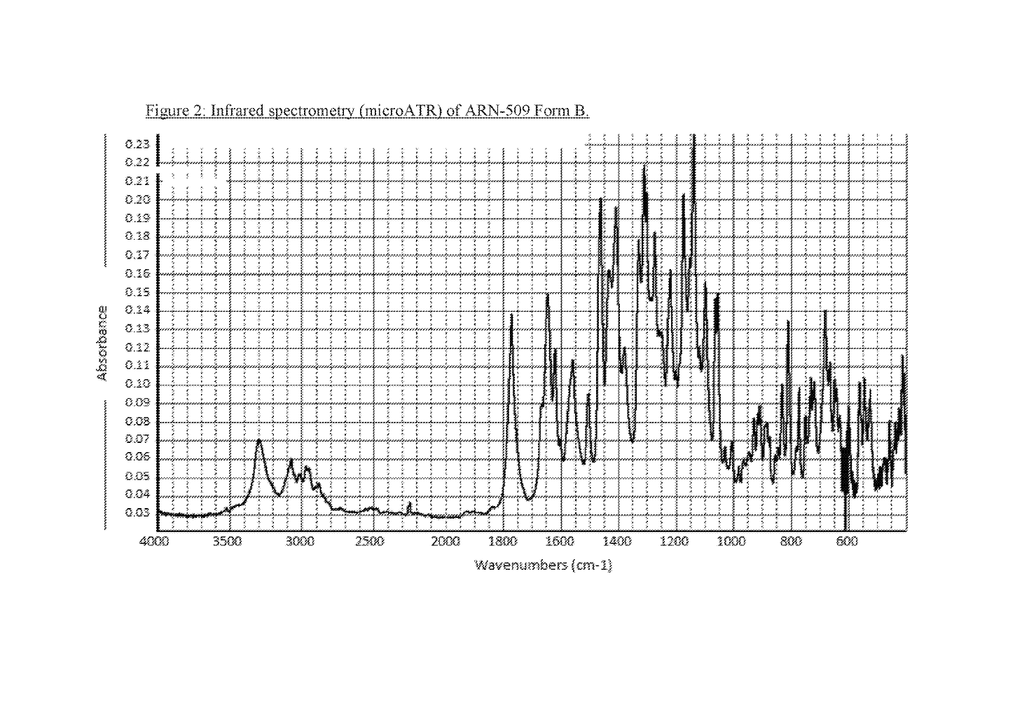

The spectrum of ARN-509 Form B is shown in FIG. 2.

Differential Scanning Calorimetry (DSC)

The compound was transferred into a standard aluminum TA-Instrument sample pan. The sample pan was closed with the appropriate cover and the DSC curve was recorded on a TA-Instruments Q1000 MTDSC equipped with a RCS cooling unit, using the following parameters:

initial temperature: 25° C.

heating rate: 10° C./min

final temperature: 250° C.

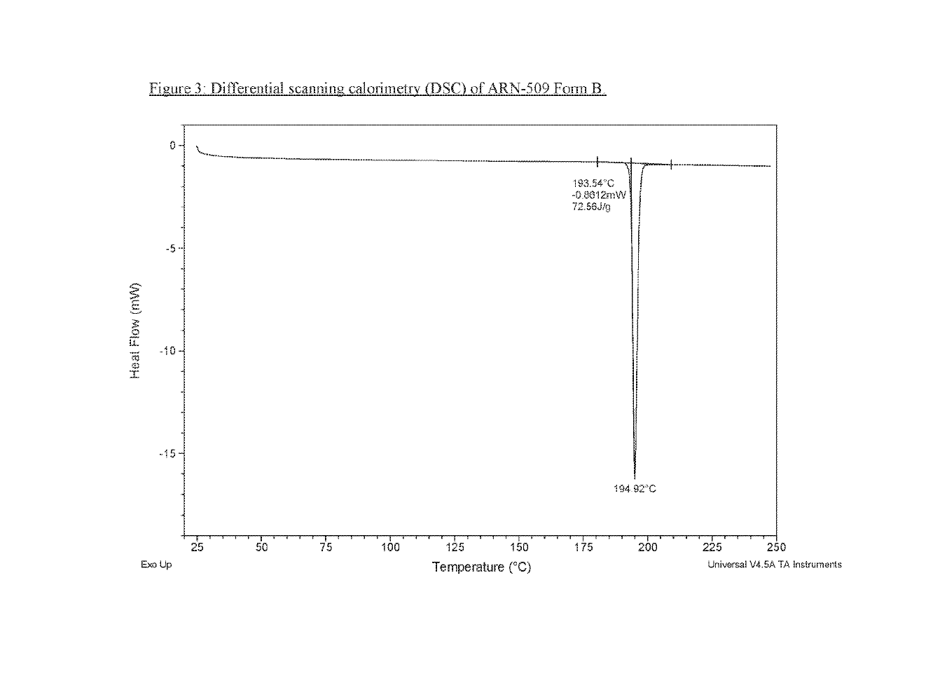

The DSC curve of ARN-509 Form B shows the melting of the product at 194.9° C. with a heat of fusion of 73 J/g. See FIG. 3.

### Example 3

### Example 3.1: Preparation of a Solid Dispersion of ARN-509:HPMCAS LG 1:3

| | | |
|---|---|---|
| ARN-509 | 2,500 | g |
| HPMC-AS LG | 7,500 | g |
| Dichloromethane, ^{a} | 76,000 | g |
| Methanol ^{a} | 114,000 | g |

^{a} Removed during processing

The dichloromethane and methanol were transferred into a suitable container and stirring was started. Under continuous stirring ARN-509 Form B was added to the solvent mixture and stirred until dissolved. HPMCAS was added to the solution and stirred overnight. A yellowish viscous turbid mixture was obtained. The mixture was filtered inline through a GRID filter. The mixture was spray dried using a suitable spray dryer, e.g. Niro A/S PSD3 with a high pressure nozzle with the following parameters: feed flow of 75 kg/hour, outlet temperature of 46° C. and a condenser temperature of −9° C.

The spray dried product (SDP) was dried in a suitable dryer, e.g. tray dryer using vacuum, nitrogen flow and a drying temperature of 40° C.

### Example 3.2: Preparation of Abiraterone Acetate Granulate

| Abiraterone acetate | 250 | mg |
|---|---|---|
| Lactose Monohydrate | 126.6 | mg |
| Croscarmellose sodium | 11.2 | mg |
| Hypromellose | 8.4 | mg |
| Sodium lauryl sulfate | 2.8 | |
| Purified Water ^{a} | 280 | mg |

^{a} Removed during processing

Lactose monohydrate, abiraterone acetate and croscarmellose sodium were charged into a bin and blended using a suitable blender to obtain a preblend.

Purified water, hypromellose and sodium lauryl sulfate were transferred into a suitable container and stirred until dissolved using a suitable mixer to obtain a binder solution. The preblend was transferred into a fluid bed granulator and the binder solution was sprayed on the preblend. The obtained granulate was dried during fluidization. The dried granules were screened.

### Example 3.3: Preparation of Tablets Comprising a Solid Dispersion of ARN-509:HPMCAS 1:3 and Abiraterone Acetate

| | | |
|---|---|---|
| Spray dried powder (SDP) of Example 3.1 | 240 | mg |
| Abiraterone acetate Granulate of Example 3.2 | 399 | mg |
| Colloidal Anhydrous Silica | 10 | mg |
| Croscarmellose sodium | 50 | mg |
| Sodium Lauryl Sulfate | 25.2 | mg |
| Silicified Microcrystalline Cellulose | 265.8 | mg |
| Magnesium stearate ^{a} | 10 | mg |

^{a} Vegetable grade for 1 tablet (1000 mg)

The ARN-509 SDP and the screened croscarmellose sodium, silicified microcrystalline cellulose, sodium lauryl sulfate, and colloidal anhydrous silica were added to the abiraterone acetate granules and blended using a suitable blender. Screened magnesium stearate was added and blended using a suitable blender. The blend was compressed into tablets (containing 60 mg of ARN 509 and 250 mg of abiraterone acetate) using a suitable tablet press.

### Example 3.4: Preparation of Coated Tablets

| | | |
|---|---|---|
| Abiraterone acetate + ARN-509 (250 mg/60 mg) | 1000 | tablets |
| Tablets (see Example 3.3) | | |
| Opadry II 85F18422 white coating powder | 30 | g |
| Purified Water ^{a} | 120 | g |

^{a} Removed during processing per batch of 1000 tablets

The coating powder was suspended in purified water and the coating suspension was sprayed on the tablets using a suitable coater.

### Example 3.5: Preparation of Coated Tablets

| | | |
|---|---|---|
| Abiraterone acetate + ARN-509 (250 mg/60 mg) | 1000 | tablets |
| Tablets (see Example 3.3) | | |
| Opadry II 32F220009 yellow coating powder | 30 | g |
| Purified Water ^{a} | 220 | g |

^{a} Removed during processing per batch of 1000 tablets

The coating powder was suspended in purified water and the coating suspension was sprayed on the tablets using a suitable coater.

### Example 4

### Example 4.1: Preparation of Tablets Comprising a Solid Dispersion of ARN-509:HPMCAS 1:3 and Abiraterone Acetate

| | | |
|---|---|---|
| Spray dried powder (SDP) of Example 3.1 | 240 | mg |
| Abiraterone acetate granulate of Example 3.2 | 399 | mg |
| Colloidal Anhydrous Silica | 10 | mg |
| Croscarmellose sodium | 50 | mg |
| Sodium Lauryl Sulfate | 12.5 | mg |
| Silicified Microcrystalline Cellulose | 278.5 | mg |
| Magnesium stearate ^{a} | 10 | mg |

^{a} Vegetable grade for 1 tablet (1000 mg)

The tablets were prepared in an analoguous manner as described above for Example 3.

### Example 4.2: Preparation of Coated Tablets

| | | |
|---|---|---|
| Abiraterone acetate + ARN-509 | 1000 | tablets |
| (250 mg/60 mg) Tablets (see Example 4.1) | | |
| Opadry II 85F18422 white coating powder | 30 | g |
| Purified Water ^{a} | 120 | g |

^{a} Removed during processing per batch of 1000 tablets

The tablets were coated in an analoguous manner as described above for Example 3.

### Example 5

### Example 5.1: Preparation of Tablets Comprising a Solid Dispersion of ARN-509:HPMCAS 1:3 and Abiraterone Acetate

| | | |
|---|---|---|
| Spray dried powder (SDP) of Example 3.1 | 240 | mg |
| Abiraterone acetate granulate of Example 3.2 | 399 | mg |
| Colloidal Anhydrous Silica | 10 | mg |
| Croscarmellose sodium | 50 | mg |
| Sodium Lauryl Sulfate | 7 | mg |
| Silicified Microcrystalline Cellulose | 284 | mg |
| Magnesium stearate ^{a} | 10 | mg |

^{a} Vegetable grade for 1 tablet (1000 mg)

The tablets were prepared in an analoguous manner as described above for Example 3.

### Example 5.2: Preparation of Coated Tablets

| | | |
|---|---|---|
| Abiraterone acetate + ARN-509 | 1000 | tablets |
| (250 mg/60 mg) Tablets (see Example 5.1) | | |
| Opadry II 85F18422 white coating powder | 30 | g |
| Purified Water ^{a} | 120 | g |

^{a} Removed during processing per batch of 1000 tablets

The tablets were coated in an analoguous manner as described above for Example 3.

### Example 6

### Example 6.1: Preparation of Abiraterone Acetate Granulate

| | | |
|---|---|---|
| Abiraterone acetate | 333.3 | mg |
| Lactose Monohydrate | 168.8 | mg |
| Croscarmellose sodium | 14.9 | mg |
| Hypromellose | 11.2 | mg |
| Sodium lauryl sulfate | 3.7 | mg |
| Purified Water ^{a} | 373.3 | mg |

^{a} Removed during processing

The abiraterone granulate was prepared in an analoguous manner as described above for Example 3.

### Example 6.2: Preparation of Tablets Comprising a Solid Dispersion of ARN-509:HPMCAS 1:3 and Abiraterone Acetate

| | | |
|---|---|---|
| Spray dried powder (SDP) of Example 3.1 | 320 | mg |
| Abiraterone acetate granulate of Example 6.1 | 532 | mg |
| Colloidal Anhydrous Silica | 12.0 | mg |
| Croscarmellose sodium | 60 | mg |
| Sodium Lauryl Sulfate | 33.6 | mg |
| Silicified Microcrystalline Cellulose | 230.4 | mg |
| Magnesium stearate ^{a} | 12.0 | mg |

^{a} Vegetable grade for 1 tablet (circa 1200 mg)

The tablets were prepared in an analoguous manner as described above for Example 3.

### Example 6.3: Preparation of Coated Tablets

| | | |
|---|---|---|
| Abiraterone acetate + ARN-509 | 1000 | tablets |
| (333.3 mg/80 mg) Tablets (see Example 6.2) | | |
| Opadry II 85F18422 white coating powder | 36 | g |
| Purified Water ^{a} | 144 | g |

^{a} Removed during processing per batch of 1000 tablets

The tablets were coated in an analoguous manner as described above for Example 3.

### Example 7

Description of solid dispersions of ARN-509 that can be used in the pharmaceutical formulations of the invention.

### Example 7.1: Preparation of a Solid Dispersion of ARN-509:HPMCAS LF 1:2

| | | |
|---|---|---|
| ARN-509 | 333.33 | mg |
| HPMCAS LF | 666.67 | mg |
| Acetone ^{a} | 19000 | mg |

^{a} Removed during processing (the reported amounts are for 1 g of SDP (spray dried product))

The acetone was transferred into a suitable container, and HPMCAS and ARN-509 Form B were added. After mixing the ingredients using a suitable mixer, the mixture was spray dried using a suitable spray dryer, e.g. Buchi mini spray dryer with the following parameters: spray rate in the range from 6.2-6.7 gram/minute, outlet temperature in the range from 46° C.-49° C. and a condenser temperature in the range from −18° C. to −21° C.

The spray dried product (SDP) was dried in a suitable dryer, e.g. tray dryer using vacuum, nitrogen flow and a drying temperature of 40° C.

### Example 7.2: Preparation of a Solid Dispersion of ARN-509:HPMCAS LF 1:3 by Hot Melt Extrusion (HME)

| | |
|---|---|
| ARN-509 | 250 mg |
| HPMCAS LF | 750 mg |

(the reported amounts are for 1 g of HME product)

The HPMCAS and ARN-509 Form B were blended in a suitable recipient using a suitable blender. Hot melt extrusion was performed in a Haake extruder, flush mode, maximum temperature 180° C., screw speed 50 rpm. The hot melt extrudate was collected and milled in a suitable mill. The milled hot melt extrudate was sieved using a suitable sieve (250 μm).

### Example 7.3: Preparation of a Solid Dispersion of ARN-509:HPMCAS LF 1:3 SDP

| | | |
|---|---|---|
| ARN-509 | 250.0 | mg |
| HPMCAS LF | 750.0 | mg |
| Acetone ^{a} | 19000.0 | mg |

^{a} Removed during processing (the reported amounts are for 1 g of SDP (spray dried product))

The acetone was transferred into a suitable container and HPMCAS LF and ARN-509 Form B were added. After mixing the ingredients using a suitable mixer, the mixture was spray dried using a suitable spray dryer, e.g. Buchi mini spray dryer with the following parameters: spray rate in the range from 5.9-6.6 gram/minute, outlet temperature in the range from 46° C.-49° C. and a condenser temperature in the range from −15° C. to −21° C.

The spray dried product (SDP) was dried in a suitable dryer, e.g. tray dryer using vacuum, nitrogen flow and a drying temperature of 40° C.

Stability Tests Performed on Powders of Example 3.1 and 7.2

The tests were performed on the powders packed in LDPE/Alu bags.

1. Appearance Testing

A visual examination was performed on the powder of Example 3.1 and Example 7.2 stored under different storage conditions as indicated in table a1 and a2 below.

The results are reported in the table a1 and a2 below.

2. Water Content

The water content was determined by means of a vaporized coulometric Karl Fischer determination in accordance with USP/Ph. Eur.

Powder of Example 3.1 or Example 7.2 was stored as indicated in table a1 or a2 below.

About 50.00 mg (±5.00 mg) of the sample was weighted accurately into a vial and the vial was crimped securely.

The results are reported in table a1 and a2 below.

The following instrumentation, reagents and solutions and parameters were used.

Instrumentation

Coulometer: 831 KF Coulometer Metrohm

Oven: 774 Sample Oven Processor Metrohm

Generator electrode: Electrode with diapraghm Metrohm 6.0344.100

Indicator electrode: Double Pt-wire electrode Metrohm 6.0341.100

Reagents and Solutions

Anode solution: Hydranal Coulomat AG Oven (Fluka 34739)

Cathode solution: Hydranal Coulomat CG (Fluka 34840)

Water standard: Hydranal Water Standard 1.00 (Fluka 34828)

Oven Parameters

Carrier gas: N2

Flow rate: Setpoint 60 mL/min
Read out value minimum 20 mL/min

Oven temperature: 120° C.

Coulometer Parameters

Titration Parameters

Extr. time: 60 s

Drift correction: Auto

Start Conditions

Pause: 60 s

Start drift: maximum 12 μg/min

Time cond. OK: 10 s

Stop Parameters

Rel. drift: 5 μg/min

Alternative coulometer parameters may be used provided system suitability requirements are met.

3. pXRD Testing for the Detection of Crystalline ARN-509

The physical stability of powder of Example 3.1 and Example 7.2 stored under different storage conditions was followed up using powder X-Ray diffraction. The XRD pattern of the powder was compared to the XRD pattern of the corresponding powder measured at time zero (amorphous product).

The powder was brought on to the zero background sample holder. A X-ray measurement of the sample was performed.

The results are reported in table a1 and a2 below.

The following instrumentation and parameters were used.

Instrumentation

Pananalytical X'Pert PRO MPD diffractometer PW3050/60

X-ray tube Cu LFF PW3373/10

Detector: X'Celerator

Sample stage: spinner

Sample holder: zero background sample holder

Instrument settings

Spinner revolution time: 1 rps

Generator voltage: 45 kV

Generator current: 40 mA

Optical components in X-ray beam path

Incident beam path:

Programmable divergence slit: irradiated length 15 mm

Soller slit: 0.04 rad

Beam mask: 15 mm

Anti-scatter slit: 1°

Beam knife+

Diffracted Beam Path:

Programmable Anti-scatter slit: 1°

Soller slit: 0.04 rad

Filter: Ni

Instrument Parameters

Geometry: Bragg-Brentano

Radiation: CuKα

Step size: 0.02°

Scan range: from 3° 2θ to 50° 2θ

Counting time per step: 60 sec

TABLE a1
Test conditions and results for powders of Example
3.1 stored in LDPE/Alu Bags-appearance and water
content and crystallinity results

| Parameter | | | | |
|---|---|---|---|---|
| | Storage | Appearance ^{a} | | |
| Storage | time | Visual | Water | |
| condition | (months) | examination | content (%) | Crystallinity |
| | Initial | Pass | 0.6 | Amorphous |
| | | | | product |
| 30° C./75% RH | 6 | Pass | 1.0 | Amorphous |
| | | | | product |
| | 9 | Pass | 1.0 | Amorphous |
| | | | | product |
| | 12 | Pass | 1.7 | Amorphous |
| | | | | product |

^{a} Pass:White to light yellow, fine to granular powder

TABLE a2
Test conditions and results for powders of Example
7.2 stored in LDPE/Alu Bags-appearance and water
content and crystallinity results

| Parameter | | | | |
|---|---|---|---|---|
| | Storage | Appearance ^{a} | | |
| Storage | time | Visual | Water | |
| condition | (months) | examination | content (%) | Crystallinity |
| | Initial | Pass | 2.3 | Amorphous |
| | | | | product |
| 30° C./75% RH | 6 | Pass | 2.0 | Amorphous |
| | | | | product |

^{a} Pass:White to light yellow-brownish, fine to granular powder

For the appearance, no substantial stability related changes were observed during storage of the drug product intermediate powder at the different storage conditions.

For the water content, no substantial stability related changes were observed during storage of the drug product intermediate powder at the different storage conditions.

For the crystallinity, no substantial stability related changes were observed during storage of the drug product at the different storage conditions.

4. Assay of ARN-509-Chromatographic Purity

The concentration of ARN-509 and its degradation products in powders of Example 3.1 and Example 7.2 stored under different storage conditions were determined by gradient Reversed-Phase UHPLC with UV Detection.

Powders were stored as indicated in table b1 and b2 below.

Approximately 240.00 mg powder was weighted accurately into a 250-mL volumetric flask. Approximately 125 mL acetonitrile was added by graduated cylinder and the whole was shaken mechanically for 30 minutes and diluted to volume with water till approximately 1 cm under the marker. The whole was shaked up manually vigorously. The sample solution was allowed to equilibrate to ambient temperature and was diluted to volume with water. Just before filtering, the volumetric flask was shaked up manually vigorously. The sample solution was filtered through a chemical resistant 0.2 μm filter. The first 3 mL filtrate were discarded into a waste container, not back into the volumetric flask.

The sample solution is stable for 4 days, if stored in refrigerator, protected from light (closed cabinet).

The results are reported in table b1 and b2 below.

The following solutions and instrumentation and parameters were used.

Mobile Phases

Mobile Phase A

10 mM NH₄Ac+0.1% TFA/Acetonitrile (90/10, v/v).

Mobile Phase B

Acetonitrile

UHPLC Conditions for Identification, Assay and Chromatographic Purity

Column: Acquity BEH C18, 150 mm length×2.1 mm i.d., 1.7 μm particle size

Column Temperature: 45° C.

Auto-Sampler Temperature: 5° C.

Flow Rate: 0.40 mL/min

Detection: UV

Wavelength: 268 nm

Injection Volume: 3 μL

Data Collection Time: 35 minutes

Analysis Run Time: 40 minutes

A linear gradient was programmed as demonstrated in the below table.

Linear Gradient Program

| Time | A | B |
|---|---|---|
| (min) | (% vol) | (% vol) |
| 0 | 100 | 0 |
| 35 | 30 | 70 |
| 36 | 100 | 0 |
| 40 | 100 | 0 |

TABLE b1
Test conditions and results for powders of Example 3.1 stored
in LDPE/Alu Bags-assay and degradation products results

| | | | Degradation products (%) |
|---|---|---|---|
| | | | Total degradation products |
| | | | (sum of all degradation |
| Parameter | | | products ≥0.05% (totals |
| Storage | Storage time | Assay(%) | are calculated on |
| condition | (months) | ARN-509 | unrounded results) |
| | Initial | 99.3 | 0.066 |
| 30° C./75% RH | 6 | 98.9 | 0.11 |
| | 9 | 98.0 | 0.07 |
| | 12 | 98.2 | 0.06 |

TABLE b2
Test conditions and results for powders of Example 7.2 stored
in LDPE/Alu Bags-assay and degradation products results

| | | | Degradation products (%) |
|---|---|---|---|
| | | | Total degradation products |
| | | | (sum of all degradation |
| Parameter | | | products ≥0.05% (totals |
| Storage | Storage time | Assay(%) | are calculated on |
| condition | (months) | ARN-509 | unrounded results) |
| | Initial | 96.4 | 0.11 |
| 30° C./75% RH | 6 | 96.3 | 0.08 |

No substantial stability related changes were observed during storage of the drug product intermediate powders at the different storage conditions.

5. Water Activity

The water activity was determined with a Novasina a_{w}-meter.

The test was performed on powders of Example 3.1 and Example 7.2.

The results are reported in table c1 and c2 below. PGP45,T1

TABLE c1
Test conditions and results for powders of Example
3.1 stored in LDPE/Alu Bags-Water activity results

Parameter

| Storage | Storage time | |
|---|---|---|
| condition | (months) | Water activity |
| | Initial | 0.21 |
| 30° C./75%RH | 12 | 0.38 |

TABLE c2
Test conditions and results for powders of Example
7.2 stored in LDPE/Alu Bags -Water activity results

| Parameter | | |
|---|---|---|
| Storage | Storage time | Water |
| condition | (months) | activity |

| | |
|---|---|
| Initial | 0.44 |

Stability Tests Performed on Film-Coated Tablets of Example 3.4, Example 3.5 and Example 5.2

For tablets of Example 3.4 and 5.2, tests were performed on coated tablets stored in closed (Clic Loc closure) white HDPE (high density polyethylene) bottles (160 ml) with Desiccant (silica gel, 2×2 g) (12 tablets/bottle).

For tablets of Example 3.5, tests were performed on coated tablets stored in closed HDPE (high density polyethylene) bottles (220 cc) with Desiccant (silica gel, 4 g) (120 tablets/bottle) and without Desiccant (120 tablets/bottle).

1. Water Content

The water content was determined by means of a vaporized coulometric Karl Fischer determination in accordance with USP/Ph. Eur.

Tablets of Example 3.4, 3.5 and 5.2 were stored as indicated in table 1a, 1b, 1c and 1d below.

Tablets were grinded using a Retsch Mixer Mill at 30 Hz for 30 seconds. Immediately after grinding, about 50.00 mg of the sample was weighted accurately into a vial and the vial was crimped securely.

The results are reported in table 1a an 1b below.

The following instrumentation, reagents and solutions and parameters were used.

Instrumentation

Coulometer: 831 KF Coulometer Metrohm

Oven: 774 Sample Oven Processor Metrohm

Generator electrode: Electrode with diapraghm Metrohm

Indicator electrode: Double Pt-wire electrode Metrohm

Reagents and Solutions

Anode solution: Hydranal Coulomat AG Oven (Fluka 34739)

Cathode solution: Hydranal Coulomat CG (Fluka 34840)

Water standard: Hydranal Water Standard 1.00 (Fluka 34828)

Oven Parameters

Carrier gas: N2

Flow rate: Setpoint 60 mL/min
Read out value minimum 20 mL/min

Oven temperature: 120° C.

Coulometer Parameters

Titration Parameters

Extr. time: 60 s

Drift correction: Auto

Start Conditions

Pause: 60 s

Start drift: maximum 12 μg/min

Time cond. OK: 10 s

Stop Parameters

Rel. drift: 5 μg/min

Alternative coulometer parameters may be used provided system suitability requirements are met

TABLE 1a
Test conditions and results for tablets of Example 3.4 stored in white
HDPE bottles with Desiccant (silica gel) - water content results

| Parameter | | water |
|---|---|---|
| Storage | Storage time | content |
| condition | (months) | (% w/w) |
| Light ICH | 0 | 1.9 |
| unprotected | | |
| Light ICH | 0 | 1.6 |
| protected | | |
| 25° | 0 | 1.9 |
| C./60% RH | 1 | 1.7 |
| | 4 | 1.7 |
| | 6 | 1.8 |
| 30° | 1 | 1.7 |
| C./75% RH | 4 | 1.8 |
| | 6 | 1.8 |
| 40° | 1 | 1.6 |
| C./75% RH | 2 | 1.7 |
| | 6 | 2.1 |

Light ICH: integrated near UV energy not less than 200 W h/m², overall illumination not less than 1200 klux · h

TABLE 1b
Test conditions and results for tablets of Example 5.2 stored in white
HDPE bottles with Desiccant (silica gel) - water content results

| Parameter | | water |
|---|---|---|
| Storage | Storage time | content |
| condition | (months) | (% w/w) |
| Light ICH | 0 | 1.8 |
| unprotected | | |
| Light ICH | 0 | 1.7 |
| protected | | |
| 25° | 0 | 2.0 |
| C./60% RH | 1 | 1.7 |
| | 4 | 1.6 |
| | 6 | 1.7 |
| 30° | 1 | 1.6 |
| C./75% RH | 4 | 1.8 |
| | 6 | 1.9 |
| 40° | 1 | 1.7 |
| C./75% RH | 2 | 1.8 |
| | 6 | 2.2 |

Light ICH: integrated near UV energy not less than 200 W · h/m², overall illumination not less than 1200 klux · h

TABLE 1c
Test conditions and results for tablets of Example 3.5 stored in
HDPE bottles (220 cc) without Desiccant - water content results

| Parameter | | water |
|---|---|---|
| Storage | Storage time | content |
| condition | (months) | (% w/w) |
| Light ICH | 0 | 3.4 |
| unprotected | | |
| 25° | 0 | 3.3 |
| C./60% RH | 7 | 3.2 |
| 30° | 3 | 3.1 |
| C./75% RH | 7 | 3.1 |
| 40° | 1 | 3.6 |
| C./75% RH | 3 | 3.2 |
| | 5 | 3.1 |
| | 7 | 3.1 |

Light ICH: integrated near UV energy not less than 200 W · h/m², overall illumination not less than 1200 klux · h

TABLE 1d
Test conditions and results for tablets of Example 3.5 stored in HDPE
bottles with Desiccant (4 gr of silica gel) - water content results

| Parameter | | water |
|---|---|---|
| Storage | Storage time | content |
| condition | (months) | (% w/w) |
| 25° | 0 | 3.3 |
| C./60% RH | 7 | 2.8 |
| 30° | 3 | 2.8 |
| C./75% RH | 7 | 2.9 |
| 40° | 1 | 3.2 |
| C./75% RH | 3 | 3.1 |
| | 5 | 2.9 |
| | 7 | 3.0 |

Light ICH: integrated near UV energy not less than 200 W · h/m², overall illumination not less than 1200 klux · h

2. Assay of ARN-509 and Abiraterone Acetate-Chromatographic Purity

The concentration of ARN-509 and its degradation products and the concentration of abiraterone acetate and its degradation products in tablets of Example 3.4, 3.5 and 5.2 stored under different storage conditions were determined by gradient Reversed-Phase UHPLC with UV Detection.

Tablets were stored as indicated in table 2a, 2b, 2c and 2d below.

For ARN-509

Five tablets were weighted accurately. Mean tablet weight was determined. Tablets were grinded to a fine powder. An amount of homogenized powder equivalent to the mean tablet weight was accurately weighted into a 250-mL volumetric flask.

Approximately 125 mL acetonitrile/water (50/50, v/v) was added by graduated cylinder (step X) and the whole was shaken mechanically for 30 minutes and diluted to volume with acetonitrile/water (50/50, v/v). The whole was shaked up manually vigorously. Just before filtering, the volumetric flask was shaked up manually vigorously. The sample solution was filtered through a chemical resistant 0.45 μm filter. The first 3 mL filtrate was discarded into a waste container, not back into the volumetric flask. The sample solution is stable for 4 days, if stored in refrigerator, protected from light (closed cabinet). Time zero starts at the execution of step X.

The results are reported in table 2a, 2b, 2c and 2d below.

The following solutions and instrumentation and parameters were used for identification of ARN-509.

Mobile Phases

Mobile Phase A

10 mM NH₄Ac (aqueous ammonium acetate)+0.1% TFA (trifluoroacetic acid)/Acetonitrile (90/10, v/v).

Mobile Phase B

Acetonitrile

UHPLC Conditions for Identification, Assay and Chromatographic Purity

Column: Acquity BEH C18, 150 mm length×2.1 mm i.d., 1.7 μm particle size

Column Temperature: 55° C.

Auto-Sampler Temperature: 5° C.

Flow Rate: 0.40 mL/min

Detection: UV

Wavelength: 268 nm

Injection Volume: 3 μL

Data Collection Time: 35 minutes

Analysis Run Time: 40 minutes

A linear gradient was programmed as demonstrated in the below table.

Linear Gradient Program

| Time | A | B |
|---|---|---|
| (min) | (% vol) | (% vol) |
| 0 | 100 | 0 |
| 35 | 30 | 70 |
| 36 | 100 | 0 |
| 40 | 100 | 0 |

For Abiraterone Acetate

Five tablets were weighted accurately. Mean tablet weight was determined. Tablets were grinded to a fine powder. An amount of homogenized powder equivalent to V2 of the mean tablet weight was accurately weighted into a 200-mL volumetric flask. Approximately 100 mL acetonitrile/water (95/5, v/v) was added by graduated cylinder (step X) and the whole was shaken mechanically for 30 minutes and diluted to volume with acetonitrile/water (95/5, v/v). The whole was shaked up manually vigorously. The sample solution was allowed to equilibrate for at least 2 hours at ambient temperature in a closed cabinet. Just before filtering, the volumetric flask was shaked up manually vigorously. The sample solution was filtered through a chemical resistant 0.2 μm filter. The first 3 mL filtrate was discarded into a waste container, not back into the volumetric flask.

The sample solution is stable for 5 days, if stored in refrigerator, protected from light (closed cabinet). Time zero starts at the execution of step X.

The results are reported in table 2a, 2b, 2c and 2d below.

The following solutions and instrumentation and parameters were used for identification of abiraterone acetate.

Mobile Phases

Mobile Phase A

10 mM NH₄Ac (ammonium acetate) in water.

Mobile Phase B

Acetonitrile

Mobile Phase C

Ethanol

UHPLC Conditions for Identification, Assay and Chromatographic Purity

Column: Acquity BEH C18, 150 mm length×2.1 mm i.d., 1.7 μm particle size

Column Temperature: 50° C.

Auto-Sampler Temperature: Ambient

Flow Rate: 0.35 mL/min

Detection: UV

Wavelength: 254 nm

Injection Volume: 3 μL

Data Collection Time: 35 minutes

Analysis Run Time: 40 minutes

A linear gradient was programmed as demonstrated in the below table.

Linear Gradient Program

| Time | A | B | C |
|---|---|---|---|
| (min) | (% vol) | (% vol) | (% vol) |
| 0 | 50 | 20 | 30 |
| 30 | 25 | 50 | 25 |
| 35 | 0 | 20 | 80 |
| 37 | 50 | 20 | 30 |
| 40 | 50 | 20 | 30 |

TABLE 2a
Test conditions and results for tablets of Example
3.4 stored in white HDPE bottles with Desiccant (silica
gel)-assay and degradation products results

| | | | Degradation products (%) |
|---|---|---|---|
| | | | Total degradation products |
| | | | of ARN-509 (sum of |
| Parameter | | | all degradation |
| | Storage | | products ≥0.05% |
| Storage | time | Assay(%) | (totals are calculated on |
| condition | (months) | ARN-509 | unrounded results) |
| Light ICH | 0 | 100.1 | 0.10 |
| unprotected | | | |
| Light ICH | 0 | 99.6 | 0.10 |
| protected | | | |
| 25° | 0 | 99.3 | 0.06 |
| C./60% RH | 1 | 100.3 | 0.05 |
| | 4 | 97.6 | 0.05 |
| | 6 | 99.2 | <0.05 |
| 30° | 1 | 100.3 | 0.10 |
| C./75% RH | 4 | 98.8 | 0.06 |
| | 6 | 99.9 | 0.18 |
| 40° | 1 | 99.1 | 0.16 |
| C./75% RH | 2 | 99.2 | 0.23 |
| | 6 | 98.0 | 1.2 |

| | | | Degradation products (%) |
|---|---|---|---|
| | | | Total degradation products |
| | | | for aberaterone acetate (sum |
| Parameter | | | of all degradation |
| | Storage | Assay(%) | products ≥0.05% |
| Storage | time | Abiraterone | (totals are calculated |
| condition | (months) | acetate | on unrounded results) |
| Light ICH | 0 | 100.5 | <0.05 |
| unprotected | | | |
| Light ICH | 0 | 101.8 | <0.05 |
| protected | | | |
| 25° | 0* | 100.7 | <0.05 |
| C./60% RH | 1 | 100.3 | <0.05 |
| | 4 | 101.4 | 0.05 |
| | 6 | 99.1 | 0.14 |
| 30° | 1 | 99.2 | <0.05 |
| C./75% RH | 4 | 100.0 | 0.12 |
| | 6 | 97.8 | 0.57 |
| 40° | 1 | 100.9 | <0.05 |
| C./75% RH | 2 | 100.0 | 0.48 |
| | 6 | 98.4 | 1.4 |

Light ICH: integrated near UV energy not less than 200 W · h/m², overall illumination not less than 1200 klux · h
*for the initial (0 month) abiraterone acetate and degradation products determination, acetonitrile was used as dilution solvent instead of acetonitrile/water (95/5, v/v).

TABLE 2b
Test conditions and results for tablets of Example
5.2 stored in white HDPE bottles with Desiccant (silica
gel)-assay and degradation products results

| | | | Degradation products (%) |
|---|---|---|---|
| | | | Total degradation products |
| | | | of ARN-509 (sum of |
| Parameter | | | all degradation |
| | Storage | | products ≥0.05% |
| Storage | time | Assay(%) | (totals are calculated on |
| condition | (months) | ARN-509 | unrounded results) |
| Light ICH | 0 | 99.8 | 0.10 |
| unprotected | | | |
| Light ICH | 0 | 100.8 | 0.10 |
| protected | | | |
| 25° | 0 | 98.8 | 0.06 |
| C./60% RH | 1 | 99.3 | 0.10 |
| | 4 | 99.5 | 0.05 |
| | 6 | 99.7 | 0.05 |
| 30° | 1 | 98.9 | 0.10 |
| C./75% RH | 4 | 98.9 | 0.06 |
| | 6 | 98.7 | 0.16 |
| 40° | 1 | 99.7 | 0.14 |
| C./75% RH | 2 | 99.6 | 0.22 |
| | 6 | 97.4 | 0.94 |

| | | | Degradation products (%) |
|---|---|---|---|
| | | | Total degradation products (sum |
| Parameter | | | of all degradation |
| | Storage | Assay(%) | products ≥0.05% |
| Storage | time | Abiraterone | (totals are calculated on |
| condition | (months) | acetate | unrounded results) |
| Light ICH | 0 | 98.8 | <0.05 |
| unprotected | | | |
| Light ICH | 0 | 98.0 | <0.05 |
| protected | | | |
| 25° | 0* | 95.9 | <0.05 |
| C./60% RH | 1 | 100.7 | <0.05 |
| | 4 | 97.5 | <0.05 |
| | 6 | 97.0 | 0.13 |
| 30° | 1 | 99.1 | <0.05 |
| C./75% RH | 4 | 98.4 | 0.08 |
| | 6 | 95.3 | 0.42 |
| 40° | 1 | 98.0 | <0.05 |
| C./75% RH | 2 | 96.8 | 0.46 |
| | 6 | 95.3 | 1.3 |

Light ICH: integrated near UV energy not less than 200 W · h/m², overall illumination not less than 1200 klux · h
*for the initial (0 month) abiraterone acetate and degradation products determination, acetonitrile was used as dilution solvent instead of acetonitrile/water (95/5, v/v).

TABLE 2c
Test conditions and results for tablets of Example 3.5 stored in HDPE
bottles without Desiccant-assay and degradation products results

| | | | Degradation products (%) |
|---|---|---|---|
| | | | Total degradation products |
| | | | for ARN-509 (sum of |
| Parameter | | | all degradation |
| | Storage | | products ≥0.05% |
| Storage | time | Assay(%) | (totals are calculated on |
| condition | (months) | ARN-509 | unrounded results) |
| Light ICH | 0 | 99.7 | 0.12 |
| unprotected | | | |
| 25° | 0 | 98.9 | 0.08 |
| C./60% RH | 7 | 100.2 | 0.18 |
| 30° | 3 | 96.4 | 0.13 |
| C./75% RH | 7 | 98.8 | 0.22 |
| 40° | 1 | 98.9 | 0.14 |
| C./75% RH | 3 | 99.3 | 0.19 |
| | 5 | 100.2 | 0.47 |
| | 7 | 98.9 | 0.57 |

| | | | Degradation products (%) |
|---|---|---|---|
| | | | Total degradation products (sum |
| Parameter | | | of all degradation |
| | Storage | Assay(%) | products ≥0.05% |
| Storage | time | Abiraterone | (totals are calculated on |
| condition | (months) | acetate | unrounded results) |
| Light ICH | 0 | 98.2 | 0.10 |
| unprotected | | | |
| 25° | 0* | 97.7 | <0.05 |
| C./60% RH | 7 | 97.5 | 0.30 |
| 30° | 3 | 97.2 | 0.22 |
| C./75% RH | 7 | 98.4 | 0.50 |
| 40° | 1 | 99.2 | 0.23 |
| C./75% RH | 3 | 97.8 | 0.44 |
| | 5 | 97.0 | 0.78 |
| | 7 | 96.0 | 0.88 |

Light ICH: integrated near UV energy not less than 200 W · h/m², overall illumination not less than 1200 klux · h
*for the initial (0 month) abiraterone acetate and degradation products determination, acetonitrile was used as dilution solvent instead of acetonitrile/water (95/5, v/v).

TABLE 2d
Test conditions and results for tablets of Example
3.5 stored in HDPE bottles with Desiccant (4 gr silica
gel)-assay and degradation products results

| | | | Degradation products (%) |
|---|---|---|---|
| | | | Total degradation products |
| | | | for ARN-509 (sum of |
| Parameter | | | all degradation |
| | Storage | | products ≥0.05% |
| Storage | time | Assay(%) | (totals are calculated on |
| condition | (months) | ARN-509 | unrounded results) |
| 25° | 0 | 98.9 | 0.08 |
| C./60% RH | 7 | 99.2 | 0.12 |
| 30° | 3 | 98.8 | 0.13 |
| C./75% RH | 7 | 99.1 | 0.22 |
| 40° | 1 | 98.4 | 0.13 |
| C./75% RH | 3 | 99.8 | 0.19 |
| | 5 | 99.1 | 0.43 |
| | 7 | 97.5 | 0.55 |

| | | | Degradation products (%) |
|---|---|---|---|
| | | | Total degradation products |
| Parameter | | | (sum of all degradation |
| | Storage | Assay(%) | products ≥0.05% |
| Storage | time | Abiraterone | (totals are calculated on |
| condition | (months) | acetate | unrounded results) |
| 25° | 0* | 97.7 | <0.05 |
| C./60% RH | 7 | 98.9 | 0.27 |
| 30° | 3 | 97.8 | 0.20 |
| C./75% RH | 7 | 96.3 | 0.51 |
| 40° | 1 | 97.7 | 0.20 |
| C./75% RH | 3 | 97.1 | 0.45 |
| | 5 | 96.8 | 0.69 |
| | 7 | 96.1 | 0.91 |

Light ICH: integrated near UV energy not less than 200 W · h/m², overall illumination not less than 1200 klux · h
*for the initial (0 month) abiraterone acetate and degradation products determination, acetonitrile was used as dilution solvent instead of acetonitrile/water (95/5, v/v).

3. Dissolution

The dissolution test was performed using Paddle Apparatus (USP type 2, Ph. Eur., JP.) at 75 rpm in 900 mL of 0.25% (w/v) sodium lauryl sulfate (SLS) in 0.05 M sodium phosphate buffer pH 4.5.

Samples were taken by Distek® sample needles with solid housing and samples were filtered with Whatman® Spartan® 0.2 μm RC (regenerated cellulose) membrane 30 mm diameter filters. After filtration the samples are stable for at least 7 days at ambient conditions when stored in clear glass vials with pierced and non-pierced septum. The sample solutions cannot be stored in the refrigerator.

The determination of the quantity of ARN-509 and abiraterone acetate present in the dissolution samples was based upon a gradient ultra high performance liquid chromatographic (UHPLC) method with UV detection.

The test was performed on tablets of Example 3.4, 3.5 and 5.2 stored under different storage conditions as indicated in table 3a, 3b, 3c and 3d below.

The following instrumentation, reagents and solutions and parameters were used.

Instrumentation

Dissolution Instrument: Paddle apparatus (USP type 2, Ph. Eur., JP).

UHPLC Instrument: Waters Acquity H-Class with UV detector.

Data Acquisition System: Waters Empower.

Analytical Balance: Sensitive to 0.01 g.

Analytical Balance: Sensitive to 0.01 mg.

pH Meter: Sensitive to 0.01 pH units.

Thermometer: Sensitive to 0.1° C.

Reagents and Solutions

Reagents

Sodium dodecyl sulfate 99% purity

Sodium lauryl sulfate (SLS)

Sodium phosphate monobasic monohydrate (NaH₂PO₄.H₂O): ACS Grade.

Ammonium acetate: HPLC grade.

Acetonitrile: HPLC Grade.

Mobile Phase

Mobile Phase A: 10 mM Ammonium acetate in water

Mobile Phase B: Acetonitrile

Procedure

Dissolution Parameters

Apparatus: Paddle Apparatus (USP type 2, Ph. Eur, JP.).

Vessels: 1-L glass.

Rotation Speed: 75 rpm.

Dissolution Medium: 0.25% (w/v) SLS in 0.05 M Phosphate Buffer pH 4.5.

Volume of Medium: 900 mL.

Medium Degassing: Not Required.

Medium Replacement: Not Required.

Temperature: 37.0±0.5° C.

Sinker: Use no sinker.

Sample Introduction: Transfer 1 tablet into each dissolution vessel.

Analytical Finish—UHPLC Parameters

Conditions

Column: Acquity UHPLC® CSH C18 1.7-μm particle size, 2.1×50 mm i.d.

Column Temperature: 60±5° C.

Sample Temperature: Ambient.

Flow Rate: 0.6 mL/min.

Detection: UV at 225 nm.

Injection Volume: 2.5 μL.

Elution Mode: gradient.

Mobile Phase: Mobile Phase A: 10 mM Ammonium acetate in water
Mobile Phase B: Acetonitrile

A linear gradient was programmed as demonstrated in the below table.

Linear Gradient Program

| Time (min) | A (% vol) | B (% vol) |
|---|---|---|
| 0 | 60 | 40 |
| 1.10 | 0 | 100 |
| 1.75 | 0 | 100 |
| 3.25 | 60 | 40 |
| 4.50 | 60 | 40 |

Degas using suitable means.

Run Time (guide): 4.5 minutes.

Retention Time (guide): Approximately 1.1 minutes for ARN-509; Approximately 1.8 minutes for abiraterone acetate

Wash Solvent: Acetonitrile.

Seal Wash Solvent: 90/10 (v:v), Water:Acetonitrile.

Purge Solvent: 90/10 (v:v), Water:Acetonitrile.

Sampling Rate: 20 points/sec with filter constant normal.

TABLE 3a
Test conditions and results for tablets of Example 3.4 stored in white HDPE bottles with Desiccant (silica gel)-dissolution results
Dissolution results for abiraterone acetate

| Parameter | | | | | | | | | | |
|---|---|---|---|---|---|---|---|---|---|---|
| | Storage | Dissolution mean abiraterone acetate (%)(min-max) | | | | | | | | |
| Storage | time | 5 | 10 | 15 | 20 | 30 | 45 | 60 | 90 | 120 |
| condition | (months) | min | min | min | min | min | min | min | min | min |
| 25° C./ | 0 | 47 | 85 | 95 | 99 | 100 | 101 | 101 | 102 | 102 |
| 60% RH | | (45-49) | (79-90) | (89-99) | (94-102) | (98-103) | (99-104) | (99-104) | (99-104) | (100-104) |
| | 1 | 41 | 82 | 93 | 96 | 99 | 100 | 100 | 100 | 101 |
| | | (37-45) | (80-84) | (90-96) | (94-99) | (97-101) | (98-101) | (98-101) | (98-101) | (99-101) |
| | 4 | 44 | 82 | 92 | 96 | 98 | 99 | 100 | 100 | 100 |
| | | (42-49) | (79-87) | (88-97) | (92-100) | (96-103) | (97-103) | (97-103) | (98-103) | (98-103) |
| | 6 | 43 | 85 | 96 | 99 | 101 | 101 | 101 | 101 | 101 |
| | | (32-51) | (82-90) | (93-100) | (97-102) | (99-104) | (100-104) | (100-104) | (100-104) | (100-104) |
| 30° C./ | 1 | 44 | 85 | 97 | 100 | 102 | 102 | 103 | 103 | 103 |
| 75% RH | | (36-50) | (83-91) | (96-101) | (99-104) | (100-105) | (101-106) | (101-106) | (101-106) | (101-106) |
| | 4 | 46 | 84 | 94 | 97 | 100 | 101 | 101 | 102 | 102 |
| | | (45-48) | (82-85) | (93-94) | (96-98) | (98-101) | (99-102) | (100-102) | (101-103) | (101-103) |
| | 6 | 49 | 85 | 94 | 96 | 98 | 99 | 99 | 99 | 99 |
| | | (48-51) | (81-90) | (90-96) | (93-99) | (95-101) | (96-102) | (96-102) | (96-102) | (96-102) |
| 40° C./ | 1 | 47 | 86 | 95 | 97 | 99 | 100 | 100 | 100 | 100 |
| 75% RH | | (43-51) | (83-90) | (92-98) | (94-101) | (96-103) | (97-104) | (97-104) | (97-104) | (97-105) |
| | 2 | 53 | 89 | 96 | 97 | 99 | 99 | 100 | 100 | 100 |
| | | (50-58) | (83-94) | (90-99) | (93-101) | (94-102) | (95-103) | (95-103) | (96-103) | (96-104) |
| | 6 | 61 | 87 | 92 | 94 | 96 | 97 | 97 | 98 | 98 |
| | | (56-65) | (84-90) | (89-95) | (91-97) | (93-99) | (94-100) | (94-100) | (95-100) | (95-100) |

Dissolution results for ARN-509

| Parameter | | | | | | | | | | |
|---|---|---|---|---|---|---|---|---|---|---|
| | Storage | Dissolution mean ARN-509 (%)(min-max) | | | | | | | | |
| Storage | time | 5 | 10 | 15 | 20 | 30 | 45 | 60 | 90 | 120 |
| condition | (months) | min | min | min | min | min | min | min | min | min |
| 25° C./ | 0 | 24 | 55 | 74 | 84 | 93 | 97 | 98 | 99 | 99 |
| 60% RH | | (23-25) | (53-56) | (72-75) | (83-86) | (93-94) | (96-98) | (97-99) | (98-99) | (98-100) |
| | 1 | 20 | 51 | 71 | 81 | 91 | 95 | 96 | 97 | 97 |
| | | (17-23) | (47-53) | (68-72) | (80-82) | (90-92) | (94-97) | (95-97) | (95-98) | (96-98) |
| | 4 | 22 | 51 | 70 | 81 | 91 | 96 | 97 | 98 | 98 |
| | | (20-23) | (49-53) | (67-72) | (78-82) | (89-92) | (95-97) | (96-98) | (97-99) | (97-99) |
| | 6 | 20 | 52 | 72 | 83 | 94 | 98 | 99 | 99 | 99 |
| | | (15-24) | (49-54) | (70-74) | (82-85) | (93-94) | (97-99) | (98-100) | (98-100) | (98-100) |
| 30° C./ | 1 | 21 | 52 | 72 | 83 | 93 | 97 | 98 | 99 | 99 |
| 75% RH | | (18-23) | (49-55) | (70-75) | (82-85) | (92-95) | (96-99) | (97-100) | (98-100) | (98-100) |
| | 4 | 21 | 51 | 69 | 80 | 90 | 95 | 96 | 97 | 97 |
| | | (20-22) | (50-52) | (68-71) | (79-81) | (90-91) | (95-96) | (96-97) | (96-98) | (96-98) |
| | 6 | 24 | 54 | 73 | 83 | 93 | 97 | 98 | 99 | 99 |
| | | (22-25) | (52-56) | (71-75) | (82-85) | (92-95) | (96-99) | (97-100) | (98-100) | (98-100) |
| 40° C./ | 1 | 23 | 55 | 74 | 85 | 94 | 98 | 99 | 99 | 99 |
| 75% RH | | (20-24) | (52-57) | (71-76) | (82-86) | (92-95) | (96-99) | (97-100) | (98-100) | (98-101) |
| | 2 | 26 | 57 | 76 | 85 | 93 | 97 | 98 | 98 | 99 |
| | | (24-27) | (56-59) | (74-80) | (84-86) | (93-94) | (96-98) | (97-99) | (98-99) | (98-100) |
| | 6 | 30 | 61 | 77 | 85 | 92 | 95 | 96 | 97 | 97 |
| | | (27-32) | (59-62) | (75-78) | (83-85) | (91-92) | (94-96) | (95-98) | (95-99) | (96-98) |

TABLE 3b
Test conditions and results for tablets of Example 5.2 stored in white HDPE bottles with Desiccant (silica gel)-dissolution results
Dissolution results for abiraterone acetate

| Parameter | | | | | | | | | | |
|---|---|---|---|---|---|---|---|---|---|---|
| | Storage | Dissolution mean abiraterone acetate (%)(min-max) | | | | | | | | |
| Storage | time | 5 | 10 | 15 | 20 | 30 | 45 | 60 | 90 | 120 |
| condition | (months) | min | min | min | min | min | min | min | min | min |
| 25° C./ | 0 | 58 | 89 | 95 | 98 | 99 | 100 | 101 | 101 | 101 |
| 60% RH | | (54-61) | (86-91) | (93-98) | (96-100) | (98-101) | (99-102) | (100-104) | (100-103) | (101-103) |
| | 1 | 52 | 85 | 92 | 95 | 96 | 97 | 97 | 98 | 98 |
| | | (45-54) | (83-87) | (91-96) | (93-98) | (95-100) | (96-100) | (96-100) | (96-102) | (97-102) |
| | 4 | 59 | 90 | 96 | 97 | 99 | 99 | 99 | 99 | 99 |
| | | (56-61) | (88-92) | (93-98) | (95-100) | (97-101) | (97-101) | (97-102) | (97-102) | (97-102) |
| | 6 | 57 | 89 | 95 | 97 | 98 | 99 | 99 | 99 | 99 |
| | | (50-62) | (87-92) | (93-98) | (95-100) | (97-102) | (97-102) | (97-102) | (97-103) | (97-103) |
| 30° C./ | 1 | 54 | 89 | 96 | 99 | 100 | 101 | 101 | 102 | 102 |
| 75% RH | | (51-58) | (87-93) | (95-99) | (98-101) | (99-102) | (100-103) | (100-103) | (101-104) | (101-104) |
| | 4 | 60 | 91 | 96 | 98 | 99 | 100 | 100 | 100 | 100 |
| | | (57-63) | (91-92) | (95-97) | (97-99) | (98-100) | (98-100) | (98-100) | (98-101) | (98-101) |
| | 6 | 62 | 89 | 95 | 97 | 98 | 99 | 100 | 100 | 100 |
| | | (58-63) | (87-92) | (92-97) | (94-100) | (96-101) | (98-102) | (98-104) | (98-102) | (98-102) |
| 40° C./ | 1 | 53 | 89 | 95 | 97 | 98 | 98 | 98 | 98 | 99 |
| 75% RH | | (44-59) | (85-92) | 94-96) | (95-98) | (97-99) | (97-99) | (97-99) | (97-98) | (98-99) |
| | 2 | 60 | 88 | 93 | 95 | 97 | 97 | 98 | 98 | 98 |
| | | (57-63) | (86-92) | (91-97) | (93-98) | (95-100) | (95-101) | (96-101) | (96-101) | (96-101) |
| | 6 | 66 | 85 | 90 | 92 | 95 | 96 | 96 | 97 | 97 |
| | | (62-69) | (80-89) | (86-94) | (89-96) | (91-98) | (92-99) | (92-99) | (94-99) | (94-99) |

Dissolution results for ARN-509

| Parameter | | | | | | | | | | |
|---|---|---|---|---|---|---|---|---|---|---|
| | Storage | Dissolution mean ARN-509 (%)(min-max) | | | | | | | | |
| Storage | time | 5 | 10 | 15 | 20 | 30 | 45 | 60 | 90 | 120 |
| condition | (months) | min | min | min | min | min | min | min | min | min |
| 25° C./ | 0 | 26 | 56 | 74 | 84 | 93 | 97 | 98 | 100 | 99 |
| 60% RH | | (24-28) | (54-59) | (72-76) | (82-86) | (91-94) | (96-98) | (97-99) | (98-104) | (98-101) |
| | 1 | 23 | 54 | 72 | 82 | 91 | 95 | 96 | 97 | 97 |
| | | (18-26) | (49-56) | (70-74) | (78-84) | (87-93) | (91-97) | (93-98) | (94-99) | (95-99) |
| | 4 | 25 | 55 | 73 | 82 | 92 | 97 | 98 | 98 | 98 |
| | | (23-27) | (54-57) | (71-74) | (81-83) | (90-93) | (94-97) | (95-98) | (96-99) | (96-99) |
| | 6 | 25 | 55 | 73 | 83 | 93 | 97 | 98 | 99 | 99 |
| | | (21-28) | (53-58) | (71-75) | (81-85) | (91-94) | (95-98) | (96-99) | (96-100) | (96-100) |
| 30° C./ | 1 | 23 | 55 | 73 | 84 | 93 | 97 | 98 | 99 | 99 |
| 75% RH | | (21-26) | (53-56) | (72-75) | (83-85) | (92-94) | (96-98) | (97-99) | (97-99) | (98-100) |
| | 4 | 26 | 56 | 73 | 82 | 91 | 95 | 96 | 97 | 97 |
| | | (23-29) | (53-58) | (71-74) | (81-83) | (90-91) | (94-96) | (95-97) | (96-97) | (96-98) |
| | 6 | 27 | 57 | 74 | 84 | 93 | 98 | 99 | 99 | 99 |
| | | (24-28) | (56-57) | (73-75) | (82-86) | (91-94) | (96-100) | (98-101) | (97-100) | (98-101) |
| 40° C./ | 1 | 24 | 56 | 74 | 84 | 93 | 98 | 99 | 99 | 99 |
| 75% RH | | (19-27) | (53-59) | (72-76) | (82-85) | (92-94) | (96-99) | (97-100) | (97-100) | (97-100) |
| | 2 | 26 | 57 | 75 | 84 | 93 | 97 | 98 | 99 | 99 |
| | | (24-28) | (56-59) | (74-76) | (83-85) | (93-94) | (97-98) | (98-99) | (98-99) | (98-100) |
| | 6 | 31 | 60 | 76 | 84 | 92 | 96 | 97 | 98 | 98 |
| | | (29-33) | (58-61) | (74-78) | (82-86) | (91-94) | (95-98) | (96-99) | (97-100) | (97-99) |

TABLE 3c
Test conditions and results for tablets of Example 3.5 stored in HDPE bottles without Desiccant-dissolution results
Dissolution results for abiraterone acetate

| Parameter | | | | | | | | | | | | |
|---|---|---|---|---|---|---|---|---|---|---|---|---|
| | Storage | Dissolution mean abiraterone acetate (%)(min-max) | | | | | | | | | | |
| Storage | time | 5 | 10 | 15 | 20 | 25 | 30 | 45 | 60 | 90 | 120 | 150 |
| condition | (months) | min | min | min | min | min | min | min | min | min | min | min |
| 25° C./ | 0 | 59 | 88 | 95 | 97 | 98 | 99 | 100 | 100 | 100 | 100 | 100 |
| 60% RH | | (49-69) | (87-90) | (93-96) | (95-99) | (96-101) | (97-102) | (98-103) | (98-102) | (98-103) | (98-103) | (98-103) |
| | 7 | 66 | 88 | 93 | 96 | 97 | 98 | 99 | 99 | 99 | 99 | 99 |
| | | (56-71) | (87-90) | (91-95) | (93-97) | (94-99) | (95-100) | (96-101) | (96-101) | (96-101) | (96-101) | (96-102) |
| 30° C./ | 3 | 65 | 89 | 94 | 96 | 98 | 98 | 99 | 100 | 100 | 100 | 100 |
| 75% RH | | (62-68) | (86-91) | (91-96) | (93-99) | (94-100) | (95-101) | (96-102) | (99-102) | (96-102) | (96-103) | (96-103) |
| | 7 | 65 | 86 | 92 | 94 | 96 | 97 | 98 | 98 | 98 | 99 | 99 |
| | | (58-70) | (84-88) | (89-94) | (92-96) | (94-98) | (95-98) | (96-99) | (97-100) | (97-100) | (98-101) | (98-101) |
| 40° C./ | 1 | 69 | 88 | 93 | 95 | 97 | 97 | 99 | 99 | 99 | 99 | 99 |
| 75% RH | | (61-75) | (85-91) | (90-96) | (93-99) | (95-100) | (95-101) | (97-103) | (96-103) | (96-103) | (97-103) | (96-103) |
| | 3 | 72 | 88 | 92 | 94 | 96 | 96 | 98 | 98 | 98 | 98 | 98 |
| | | (68-76) | (87-89) | (91-94) | (94-96) | (95-97) | (96-98) | (96-100) | (96-100) | (96-99) | (96-101) | (97-100) |
| | 5 | 67 | 85 | 90 | 92 | 94 | 95 | 96 | 96 | 97 | 97 | 97 |
| | | (58-73) | (81-87) | (87-93) | (90-95) | (91-97) | (92-98) | (94-99) | (94-99) | (95-99) | (95-99) | (95-99) |
| | 7 | 70 | 85 | 90 | 93 | 94 | 95 | 96 | 97 | 97 | 97 | 98 |
| | | (67-73) | (83-87) | (88-92) | (91-94) | (92-96) | (94-97) | (95-99) | (95-99) | (96-99) | (96-99) | (96-99) |
| Light | 0 | 57 | 88 | 94 | 96 | 97 | 98 | 99 | 99 | 99 | 99 | 99 |
| ICH un- | | (53-63) | (86-89) | (93-94) | (95-97) | (97-98) | (97-99) | (98-100) | (98-101) | (98-101) | (98-101) | (98-101) |
| protected | | | | | | | | | | | | |

Dissolution results for ARN-509

| Parameter | | | | | | | | | | | | |
|---|---|---|---|---|---|---|---|---|---|---|---|---|
| | Storage | Dissolution mean ARN-509 (%)(min-max) | | | | | | | | | | |
| Storage | time | 5 | 10 | 15 | 20 | 25 | 30 | 45 | 60 | 90 | 120 | 150 |
| condition | (months) | min | min | min | min | min | min | min | min | min | min | min |
| 25° C./ | 0 | 31 | 65 | 83 | 92 | 96 | 98 | 101 | 101 | 101 | 101 | 101 |
| 60% RH | | (25-38) | (61-69) | (81-86) | (91-93) | (95-97) | (98-99) | (100-102) | (99-102) | (100-102) | (100-102) | (100-103) |
| | 7 | 36 | 69 | 85 | 92 | 95 | 97 | 98 | 98 | 98 | 99 | 99 |
| | | (28-40) | (64-72) | (81-87) | (90-93) | (93-96) | (95-98) | (97-99) | (98-99) | (98-99) | (98-99) | (98-99) |
| 30° C./ | 3 | 35 | 69 | 85 | 92 | 96 | 98 | 99 | 100 | 99 | 100 | 100 |
| 75% RH | | (32-38) | (66-70) | (83-86) | (91-93) | (95-97) | (97-99) | (98-101) | (99-100) | (98-101) | (99-101) | (99-101) |
| | 7 | 37 | 70 | 85 | 92 | 95 | 97 | 98 | 98 | 99 | 99 | 99 |
| | | (33-40) | (68-73) | (83-88) | (90-95) | (93-98) | (95-99) | (96-100) | (96-100) | (96-100) | (97-101) | (97-101) |
| 40° C./ | 1 | 40 | 71 | 86 | 93 | 96 | 97 | 99 | 99 | 99 | 100 | 100 |
| 75% RH | | (35-45) | (68-75) | (84-88) | (91-94) | (95-97) | (96-98) | (98-100) | (98-100) | (98-101) | (99-101) | (98-101) |
| | 3 | 42 | 73 | 87 | 93 | 96 | 97 | 98 | 98 | 99 | 99 | 99 |
| | | (39-46) | (71-76) | (85-88) | (93-94) | (95-97) | (97-98) | (98-100) | (98-99) | (98-99) | (98-100) | (98-100) |
| | 5 | 38 | 70 | 85 | 92 | 95 | 96 | 97 | 98 | 98 | 98 | 98 |
| | | (33-41) | (67-72) | (82-87) | (90-94) | (93-97) | (95-99) | (96-100) | (97-100) | (97-100) | (97-100) | (97-100) |
| | 7 | 42 | 73 | 87 | 93 | 96 | 97 | 98 | 98 | 98 | 99 | 99 |
| | | (38-45) | (70-76) | (84-89) | (91-95) | (95-98) | (95-99) | (96-100) | (96-100) | (96-100) | (97-101) | (98-101) |
| Light | 0 | 31 | 65 | 83 | 91 | 96 | 97 | 99 | 100 | 100 | 100 | 100 |
| ICH un- | | (28-35) | (62-69) | (81-85) | (90-93) | (94-96) | (96-99) | (98-100) | (98-101) | (98-101) | (99-101) | (98-101) |
| protected | | | | | | | | | | | | |

TABLE 3d
Test conditions and results for tablets of Example 3.5 stored in HDPE bottles with Desiccant (4 g silica gel)-dissolution results
Dissolution results for abiraterone acetate

| Parameter | | | | | | | | | | | | |
|---|---|---|---|---|---|---|---|---|---|---|---|---|
| | Storage | Dissolution mean abiraterone acetate (%)(min-max) | | | | | | | | | | |
| Storage | time | 5 | 10 | 15 | 20 | 25 | 30 | 45 | 60 | 90 | 120 | 150 |
| condition | (months) | min | min | min | min | min | min | min | min | min | min | min |
| 25° C./ | 7 | 56 | 88 | 95 | 97 | 98 | 99 | 100 | 100 | 100 | 101 | 101 |
| 60% RH | | (52-63) | (83-91) | (90-98) | (93-100) | (94-101) | (95-102) | (96-103) | (96-103) | (96-104) | (96-104) | (96-104) |
| 30° C./ | 3 | 64 | 89 | 94 | 96 | 97 | 98 | 99 | 99 | 99 | 99 | 99 |
| 75% RH | | (56-77) | (87-90) | (92-96) | (95-98) | (96-99) | (97-99) | (97-100) | (98-101) | (98-101) | (98-100) | (97-100) |
| | 7 | 60 | 86 | 91 | 93 | 95 | 95 | 96 | 97 | 97 | 97 | 97 |
| | | (49-70) | (84-88) | (88-94) | (90-96) | (91-97) | (92-98) | (93-99) | (93-100) | (94-100) | (94-100) | (95-101) |
| 40° C./ | 1 | 68 | 88 | 93 | 95 | 97 | 97 | 98 | 99 | 99 | 99 | 99 |
| 75% RH | | (61-73) | (83-90) | (89-95) | (92-98) | (93-99) | (94-99) | (96-100) | (97-100) | (96-101) | (97-101) | (97-101) |
| | 3 | 68 | 86 | 91 | 94 | 95 | 96 | 97 | 97 | 97 | 97 | 97 |
| | | (65-74 | (84-87) | (90-93) | (92-94) | (94-96) | (94-97) | (95-98) | (95-98) | (95-98) | (95-98) | (95-98) |
| | 5 | 63 | 85 | 90 | 93 | 94 | 95 | 96 | 97 | 97 | 97 | 97 |
| | | (59-71) | (81-88) | (87-93) | (90-95) | (92-97) | (93-98) | (94-98) | (95-98) | (95-99) | (95-99) | (95-99) |
| | 7 | 67 | 85 | 90 | 93 | 94 | 95 | 97 | 97 | 98 | 98 | 98 |
| | | (62-73) | (83-87) | (89-91) | (92-94) | (93-95) | (95-96) | (96-98) | (96-99) | (96-99) | (96-100) | (96-100) |

Dissolution results for ARN-509

| Parameter | | | | | | | | | | | | |
|---|---|---|---|---|---|---|---|---|---|---|---|---|
| | Storage | Dissolution mean ARN-509 (%)(min-max) | | | | | | | | | | |
| Storage | time | 5 | 10 | 15 | 20 | 25 | 30 | 45 | 60 | 90 | 120 | 150 |
| condition | (months) | min | min | min | min | min | min | min | min | min | min | min |
| 25° C./ | 7 | 29 | 64 | 82 | 90 | 95 | 97 | 99 | 99 | 99 | 99 | 100 |
| 60% RH | | (25-33) | (60-69) | (79-85) | (89-92) | (93-96) | (95-98) | (98-100) | (98-100) | (98-101) | (99-101) | (99-101) |
| 30° C./ | 3 | 33 | 67 | 83 | 91 | 95 | 97 | 99 | 99 | 99 | 99 | 99 |
| 75% RH | | (29-41) | (65-71) | (82-85) | (90-92) | (94-96) | (96-98) | (98-100) | (98-101) | (98-100) | (98-100) | (98-100) |
| | 7 | 33 | 67 | 83 | 90 | 94 | 95 | 97 | 97 | 98 | 98 | 98 |
| | | (25-41) | (61-71) | (80-85) | (88-92) | (92-95) | (94-96) | (96-98) | (96-98) | (96-98) | (96-98) | (98-99) |
| 40° C./ | 1 | 37 | 69 | 85 | 92 | 96 | 97 | 99 | 99 | 99 | 99 | 99 |
| 75% RH | | (35-40) | (68-71) | (84-86) | (91-93) | (95-96) | (96-98) | (97-99) | (98-100) | (98-100) | (98-100) | (98-100) |
| | 3 | 38 | 70 | 85 | 92 | 95 | 97 | 98 | 98 | 98 | 98 | 98 |
| | | (35-43) | (68-73) | (84-87) | (91-93) | (94-96) | (96-98) | (97-99) | (97-99) | (98-99) | (98-99) | (98-99) |
| | 5 | 35 | 68 | 84 | 91 | 94 | 96 | 97 | 98 | 98 | 98 | 98 |
| | | (33-40) | (66-71) | (82-86) | (90-92 | (93-95) | (95-96) | (97-98) | (97-98) | (97-99) | (97-99) | (97-99) |
| | 7 | 40 | 72 | 86 | 92 | 95 | 96 | 97 | 98 | 98 | 98 | 99 |
| | | (37-45) | (69-75) | (83-89) | (90-95) | (93-97) | (94-98) | (96-99) | (96-99) | (97-99) | (97-100) | (97-100) |

4. Content Uniformity

The Content Uniformity of ARN-509 and abiraterone acetate in tablets of example 3.4, 3.5 and 5.2 was determined by gradient Reversed-Phase UHPLC with UV Detection.

A tablet was brought into a 250 mL volumetric flask. 10 mL of water was added by using a graduated cylinder (step X) and the whole was shaken mechanically for 10 minutes. Approximately 150 mL of acetonitrile was added by using a graduated cylinder and the whole was shaken mechanically for 30 minutes and it was diluted to volume with acetonitrile till approximately 1 cm under the marker. The whole was shaked up manually vigorously. The sample solution was allowed to equilibrate to ambient temperature. The sample solution was diluted to volume with acetonitrile. 8.0 mL of the solution was transferred, using a volumetric pipette, into a 50 mL volumetric flask and diluted to volume with acetonitrile. Just before filtering, the volumetric flask was shaked up manually vigorously. The sample solution was filtered through a chemical resistant 0.2 μm filter. The first 3 mL filtrate was discarded into a waste container, not back into the volumetric flask. The auto-sampler vial was filled to the appropriate height with filtrate.

The sample solution is stable for 5 days, if stored at ambient temperature, protected from light (closed cabinet). Time zero starts at the execution of step X.

The results are reported below.

The following solutions and instrumentation and parameters were used.

Mobile Phases

Mobile Phase A

10 mM NH4Ac (ammonium acetate) in water.

Mobile Phase B

Acetonitrile

UHPLC Conditions

Column: Acquity BEH C18, 50 mm length×2.1 mm i.d., 1.7 μm particle size

Column Temperature: 55° C.

Auto-Sampler Temperature: ambient

Flow Rate: 0.6 mL/min

Detection: UV

Wavelength: 242 nm for ARN-509 and 254 nm for abiraterone acetate

Injection Volume: 3 μL

Data Collection Time: 6 minutes

Analysis Run Time: 9 minutes

A linear gradient was programmed as demonstrated in the below table.

Linear Gradient Program

| Time (min) | A (% vol) | B (% vol) |
|---|---|---|
| 0 | 85 | 15 |
| 4.0 | 5 | 95 |
| 5.0 | 5 | 95 |
| 6.0 | 85 | 15 |
| 9.0 | 85 | 15 |

Content uniformity results for tablets of Example 3.4 stored in white HDPE bottles with Desiccant (silica gel):

Mean ARN-509 (Min-Max) (%): 0 months: 99.6 (98.7-101.2)

Mean abiraterone acetate (Min-Max) (%): 0 months: 101.9 (98.8-103.2)

Content uniformity results for tablets of Example 5.2 stored in white HDPE bottles with Desiccant (silica gel):

Mean ARN-509 (Min-Max) (%): 0 months: 99.4 (96.7-100.5)

Mean abiraterone acetate (Min-Max) (%): 0 months: 100.5 (97.5-104.4)

Content uniformity results for tablets of Example 3.5 stored in HDPE bottles without Desiccant:

Mean ARN-509 (Min-Max) (%): 0 months: 99.6 (98.0-101.6)

Mean abiraterone acetate (Min-Max) (%): 0 months: 98.8 (96.3-101.2)

5. pXRD Testing for the Detection of Crystalline ARN-509

The nature of the crystalline abiraterone acetate and the amourphous ARN-509 in the tablets of Example 3.5 was assessed by X-Ray diffraction. Amorphous ARN-509 spray dried powder does not show any characteristic diffraction peaks but a halo characteristic of the amourphous material.

The tablet was gently grinded using a morat and pestle. The cavity of the sample holder was filled using the back loading technique.

The diffraction peaks of the sample pattern should correspond to those of the reference diffraction pattern. A relative shifting of all peak positions (less than ±0.20 (20)) may occur when comparing the diffractograms from different samples. This could be due to the differences in sample height. The intensities of the diffraction peaks should follow the overall trend although they can vary due to several effects, i.e. preferred orientation, particle size etc.

The results are reported in table 4a and 4b below.

The following instrumentation and parameters were used.

Instrumentation

X'Pert diffractometer

X-ray tube Cu LFF

Detector: X'Celerator

Sample stage: spinner

Sample holder: cavity holder

Instrument settings

Spinner revolution: yes

Generator voltage: 45 kV

Generator current: 40 mA

Radiation type: CuKα

Geometry: Bragg-Brentano

Step size: 0.02°

Scan range: from 3° to 50° 2θ

Counting time per step: 100 s

Optical Components

Incident Beam Path:

Programmable divergence slit: irradiated length 10 mm

Soller slit: 0.04 rad

Beam mask: 10 mm

Anti-scatter slit: 1°

Diffracted Beam Path:

Anti-scatter device: present

Soller slit: 0.04 rad

Filter: Ni

TABLE 4a
Test conditions and results for tablets of Example 3.5 stored
in HDPE bottles without Desiccant -crystallinity results

| Parameter | | | |
|---|---|---|---|
| | Storage | | |
| Storage | time | Crystallinity | Crystallinity |
| condition | (months) | Abiraterone acetate | ARN-509 |
| Light ICH | 0 | Crystalline drug substance | Amorphous drug substance in |
| unprotected | | in the drug product | the drug product |
| 25° | 0 | Crystalline drug substance | Amorphous drug substance in |
| C./60% RH | | in the drug product | the drug product |
| | 7 | Crystalline drug substance | Amorphous drug substance in |
| | | in the drug product | the drug product |
| 40° | 1 | Crystalline drug substance | Amorphous drug substance in |
| C./75% RH | | in the drug product | the drug product |
| | 3 | Crystalline drug substance | Amorphous drug substance in |
| | | in the drug product | the drug product |
| | 5 | Crystalline drug substance | Amorphous drug substance in |
| | | in the drug product | the drug product |
| | 7 | Crystalline drug substance | Amorphous drug substance in |
| | | in the drug product | the drug product |
| 30° | 3 | Crystalline drug substance | Amorphous drug substance in |
| C./75% RH | | in the drug product | the drug product |
| | 7 | Crystalline drug substance | Amorphous drug substance in |
| | | in the drug product | the drug product |

TABLE 4b
Test conditions and results for tablets of Example 3.5 stored
in HDPE bottles with Desiccant -crystallinity results

| Parameter | | | |
|---|---|---|---|
| | Storage | | |
| Storage | time | Crystallinity | Crystallinity |
| condition | (months) | Abiraterone acetate | ARN-509 |
| 25° | 0 | Crystalline drug substance | Amorphous drug substance in |
| C./60% RH | | in the drug product | the drug product |
| | 7 | Crystalline drug substance | Amorphous drug substance in |
| | | in the drug product | the drug product |
| 40° | 1 | Crystalline drug substance | Amorphous drug substance in |
| C./75% RH | | in the drug product | the drug product |
| | 3 | Crystalline drug substance | Amorphous drug substance in |
| | | in the drug product | the drug product |
| | 5 | Crystalline drug substance | Amorphous drug substance in |
| | | in the drug product | the drug product |
| | 7 | Crystalline drug substance | Amorphous drug substance in |
| | | in the drug product | the drug product |
| 30° | 3 | Crystalline drug substance | Amorphous drug substance in |
| C./75% RH | | in the drug product | the drug product |
| | 7 | Crystalline drug substance | Amorphous drug substance in |
| | | in the drug product | the drug product |

It is within the knowledge of the skilled person to recognize equivalent conditions, solutions, reagents, parameters and instrumentation to the ones described above. It is within the knowledge of the skilled person to recognize appropriate reference solutions, calculation methods, suitability tests.

1. A pharmaceutical formulation comprising a pharmaceutically acceptable carrier, abiraterone acetate and a solid dispersion, said solid dispersion comprising ARN-509 and a polymer selected from HPMCAS, a poly(meth)acrylate copolymer, and mixtures thereof.

2. The pharmaceutical formulation according to claim 1 wherein the solid dispersion comprises ARN-509 and HPMCAS.

3. The pharmaceutical formulation according to claim 2 wherein the dispersion consists of ARN-509 and HPMCAS.

4. The pharmaceutical formulation according to claim 2 wherein the weight-by-weight ratio of ARN-509:HPMCAS in the solid dispersion is in the range from 1:1 to 1:5.

5. The pharmaceutical formulation according to claim 4 wherein the weight-by-weight ratio of ARN-509:HPMCAS in the solid dispersion is 1:3.

6. The pharmaceutical formulation according to claim 1 wherein ARN-509 is present in amorphous form.

7. The pharmaceutical formulation according to claim 1 wherein the dispersion is a solid solution.

8. The pharmaceutical formulation according to claim 1 wherein the HPMCAS is HPMCAS LG.

9. The pharmaceutical formulation according to claim 1 further comprising 250 mg of abiraterone acetate.

10. The pharmaceutical formulation according to claim 1 comprising 60 mg of ARN-509.

11. The pharmaceutical formulation according to claim 1 wherein the formulation is a tablet.

12. The pharmaceutical formulation according to claim 11 which is suitable for oral administration.

13. A process for preparing a pharmaceutical formulation according to claim 1 comprising the steps of:
a) preparing a solid dispersion of ARN-509 and a polymer selected from HPMCAS, a poly(meth)acrylate copolymer, and mixtures thereof;
b) preparing a granulate comprising abiraterone acetate;
c) mixing the solid dispersion of a) and the granulate of b) and a pharmaceutically acceptable carrier.

14. The process according to claim 13 wherein the solid dispersion of ARN-509 and the polymer is prepared by mixing ARN-509 and the polymer in a suitable solvent and spray drying said mixture.

15. The process according to claim 14 wherein the suitable solvent is a mixture of dichloromethane and methanol.

16. The process according to claim 15 wherein the weight:weight ratio of dichloromethane to methanol in the mixture is 4:6.

17. The process according to any one of claim 13 wherein the polymer is HPMCAS.

18. A method of treating prostate cancer in a human male comprising administering to such male a formulation of claim 1.

19. The method of claim 18 wherein the formulation is for oral administration.

20. A combination of a pharmaceutical formulation according to claim 1 and a glucocorticoid selected from the group consisting of prednisone, prednisolone, methylprednisolone, dexamethasone and pharmaceutically acceptable salts and acetates thereof.

21. The combination according to claim 20 wherein the glucocorticoid is prednisone.

22. The combination according to claim 20 wherein the glucocorticoid is prednisolone.
